SEQUENCE LISTING (27 sequences)

SEQ ID NO: 1 - protein, 25 aa, synthetic construct
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 9..18: note = This region may encompass 1-10 residues or may be absent
TKKLTLRTXXXXXXXXXXMLPHHGA

SEQ ID NO: 2 - protein, 5 aa, synthetic construct
EAAAK

SEQ ID NO: 3-4 - skipped (2 entries)

SEQ ID NO: 5 - protein, 5 aa, synthetic construct
PAPAP

SEQ ID NO: 6 - protein, 5 aa, synthetic construct
GSGGG

SEQ ID NO: 7 - protein, 7 aa, synthetic construct
KGSVLSA

SEQ ID NO: 8 - protein, 8 aa, synthetic construct
PKSALQEL

SEQ ID NO: 9 - protein, 8 aa, synthetic construct
GLALLGWG

SEQ ID NO: 10 - protein, 7 aa, synthetic construct
LGWLSAV

SEQ ID NO: 11 - protein, 9 aa, synthetic construct
WLMNYFWPL

SEQ ID NO: 12 - protein, 9 aa, synthetic construct
YLMNYLLPY

SEQ ID NO: 13 - protein, 20 aa, synthetic construct
TKKLTLRTEAAAKMLPHHGA

SEQ ID NO: 14 - protein, 18 aa, synthetic construct
TKKLTLRTAPAMLPHHGA

SEQ ID NO: 15 - protein, 18 aa, synthetic construct
TKKLTLRTGGGMLPHHGA

SEQ ID NO: 16 - protein, 20 aa, synthetic construct
TKKLTLRTPAPAPMLPHHGA

SEQ ID NO: 17 - protein, 20 aa, synthetic construct
TKKLTLRTGSGGGMLPHHGA

SEQ ID NO: 18 - protein, 22 aa, synthetic construct
TKKLTLRTKGSVLSAMLPHHGA

SEQ ID NO: 19 - protein, 23 aa, synthetic construct
TKKLTLRTPKSALQELMLPHHGA

SEQ ID NO: 20 - protein, 23 aa, synthetic construct
TKKLTLRTGLALLGWGMLPHHGA

SEQ ID NO: 21 - protein, 22 aa, synthetic construct
TKKLTLRTLGWLSAVMLPHHGA

SEQ ID NO: 22 - protein, 24 aa, synthetic construct
TKKLTLRTWLMNYFWPLMLPHHGA

SEQ ID NO: 23 - protein, 24 aa, synthetic construct
TKKLTLRTYLMNYLLPYMLPHHGA

SEQ ID NO: 24 - protein, 15 aa, synthetic construct
TKKLTLRTMLPHHGA

SEQ ID NO: 25 - protein, 8 aa, synthetic construct
TKKLTLRT

SEQ ID NO: 26 - protein, 7 aa, synthetic construct
MLPHHGA

SEQ ID NO: 27 - protein, 7 aa, unidentified
TKKTLRT

(19) United States
(12) Patent Application Publication
(10) Pub. No.: US 2026/0224446 A1
(43) Pub. Date: Aug. 6, 2026

(54) BIFUNCTIONAL PEPTIDES USEFUL FOR COLLAGEN INTRAFIBRILLAR MINERALIZATION

(71) Applicant: University of Kansas, Lawrence, KS (US)

(72) Inventors: Candan TAMERLER-BEHAR, Lawrence, KS (US); Paulette SPENCER, Lawrence, KS (US); Malcolm L SNEAD, Los Angeles, CA (US)

(73) Assignee: University of Kansas, Lawrence, KS (US)

(21) Appl. No.: 19/155,163

(22) PCT Filed: Feb. 8, 2024
(86) PCT No.: PCT/US2024/014965

§ 371 Date: Aug. 8, 2025

Related U.S. Application Data
(60) Provisional application No. 63/444,209, filed on Feb. 8, 2023.

(51) Int. Cl. A61K 6/30; C07K 14/78

(52) U.S. Cl. CPC A61K 6/30; C07K 14/78

(57) ABSTRACT

Described herein are bifunctional peptides, compositions comprising the same, and methods useful for treatment of dental caries.

### CROSS-REFERENCE TO RELATED APPLICATIONS

This application is a U.S. National Stage under 35 U.S.C. § 371 of International Patent Application No. PCT/US2024/014965, filed Feb. 8, 2024, which claims the benefit of and priority to U.S. Provisional Patent Application No. 63/444,209, filed Feb. 8, 2023, which is incorporated by reference herein in its entirety for any and all purposes.

### STATEMENT OF U.S. GOVERNMENT SUPPORT

This invention was made with government support under DE025476 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on May 22, 2024, is named 104434-0344_SL.xml and is 27,302 bytes in size.

### BACKGROUND

Dental caries is the most ubiquitous infectious disease of mankind resulting in destruction of the teeth and is recognized as a global health crisis [54]; no infectious disease is more common than dental caries (also referred to by the general population as cavities). Composite resins are widely adapted in restorative dentistry, but their short lifespan leads to a cycle of repeated replacement along with an inherent risk of pulpal injury, loss of tooth structure, and weakened tooth resulting in fracture. The leading cause of composite restoration failure is recurrent decay. In contrast to dental amalgam, composite lacks the capability to seal gaps at the interface between the restorative material and tooth structure.

The low-viscosity adhesive that bonds the composite to the tooth is intended to seal this interface, but the adhesive seal to dentin is readily damaged by chemical and mechanical stresses. The adhesive/dentin bond is a heterogeneous construct-its composition includes the hybrid layer, which ideally integrates adhesive with highly porous collagen fibers. Failure of the adhesive/dentin bond is the critical issue preventing long lasting dental restorations [4,10,68]. The weaker link resulting in recurrent decay is the hybrid layer, which integrates adhesive with the highly porous collagen fibers. Following acid-etching, demineralized collagen fibrils contain trapped water, which is extremely difficult to remove. Achieving complete enclosure of demineralized collagen fibrils by monomers to fill in and close those spaces is recognized as unattainable under clinical conditions.

### SUMMARY

In an aspect, the present technology provides a bifunctional peptide of amino acid sequence TKKLTLRT-X₁-MLPHHGA (SEQ ID NO: 1) or one or both of a pharmaceutically acceptable salt thereof and a solvate thereof, where X₁ is absent (a bond) or is a spacer of 1, 2, 3, 4, 5, 6, 7, 8, 9, or 10 amino acids.

In an aspect, a composition is provided that includes a bifunctional peptide of the present technology and a pharmaceutically acceptable carrier.

In an aspect, the present technology provides a dental adhesive composition that includes a bifunctional peptide of any disclosed herein and one or more dental adhesives.

In an aspect, a method of adhering a dental composite to a dental surface is provided where the method includes administering to the dental surface a bifunctional peptide of any embodiment disclosed herein, administering to the dental surface a composition of any embodiment disclosed herein, and/or administering to the dental surface a dental adhesive composition of any embodiment disclosed herein, to provide a treated dental surface; and contacting the dental composite with the treated dental surface. The method may, among other things, facilitate the formation of new calcium phosphate mineral layers on dental surface and/or improve adhesion by facilitating collagen infiltration.

### BRIEF DESCRIPTION OF THE DRAWINGS

FIGS. 1A-1C provide a representative human dentin profile, where FIG. 1A provides a RAMAN light microscopy image at 50× over 400 μm×140 μm area, marked regions comparing demineralized dentin (DD) to intact dentin (ID), FIG. 1B provides RAMAN spectra of DD and ID highlighted regions, and FIG. 1C provides RAMAN spectra of rat tail SC collagen on glass pre/post 60 min of ALP-driven mineralization.

FIGS. 2A-2C provide representative results of the evaluation of using of MLPHHGA (SEQ ID NO. 26; “HABP1”) to potentially direct mineralization within a adhesive/demineralized (“A/D”) dentin hybrid layer. FIG. 2A provides RAMAN imaging with spectral analysis adjacent of A/D interface pre/post ALP-driven mineralization with HABP1 present, FIG. 2B RAMAN DCA of HABP1 mineralized surface on mineral types present and corresponding spectral analysis, and FIG. 2C isolated DCA mapping of collagen, HABP1 mineral group I, HABP1 mineral group II, and full width at half maximum (FWHM) spatial mapping, according to the working examples.

FIG. 3 provides a comparison of FT-IR spectra investigating the secondary structure of collagenase-collagen-binding peptide TKKLTLRT (SEQ ID NO. 25) indicated as spectra A), hydroxyapatite-binding peptide MLPHHGA (SEQ ID NO. 26) indicated as spectra B, and a bifunctional peptide of the present technology with the sequence TKKLTLRTAPAMLPHHGA (SEQ ID NO: 14) indicated as spectra C.

FIG. 4 summarizes the results of examining mechanical properties of collagen control and peptide-functionalized collagen samples through PeakForce-QNM AFM, according to the working examples. Modulus averaged per pre-mineralization sample, error bars indicating standard deviation, and significant difference (p<0.05) is represented as *, **, ****, **** Scale bar=500 nm.

FIG. 5 summarizes the results of characterization of peptide-functionalized collagen surfaces after 20 min of ALP-driven mineralization examined through PeakForce-QNM AFM, according to the working examples, where comparison between pre- and post-mineralization per sample represented as averages over the area, error bars representing standard deviation, significant difference (p<0.05) is represented as *, **. Scale bar=1 μm.

FIGS. 6A-6D provide the results of mineral deposition on a collagen control and peptide-incorporated collagen samples as shown by SEM images with corresponding EDS spectra, where FIG. 6A is for the collagen control, FIG. 6B collagen incorporating TKKLTLRT (SEQ ID NO. 25; “CBP”), FIG. 6C collagen incorporating MLPHHGA (SEQ ID NO. 26; “HABP1”), and FIG. 6D collagen incorporating a bifunctional peptide of the present technology with the sequence TKKLTLRTAPAMLPHHGA (SEQ ID NO: 14; (“CBP-HABP1”), according to the working examples. Ca/P averages displayed below EDS spectra, calculated across minimum three unique areas per peptide-functionalized collagen sample. Scale bar is under each image, 1 mm.

FIGS. 7A-7B provide shaded surface display of mineral formation on peptide-functionalized collagen samples, via Micro-CT, after 20-min ALP-driven mineralization, magnification level 20×, where FIG. 7A provides the results of CBP-HABP guided ALP-based mineralized collagen samples and FIG. 7B provides the results of ALP-based remineralization on collagen samples, according to the working examples. Scale bar is under each image, 50 μm.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C; B and C, or the combination of A, B, and C.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As used herein, the term “peptide” refers to a polymer of amino acid residues joined by amide linkages, which may optionally be chemically modified to achieve desired characteristics. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include unnatural amino acids or residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

By “pharmaceutically acceptable” is meant a material that is not biologically or otherwise undesirable, e.g., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

Pharmaceutically acceptable salts of peptides described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

The peptides of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

As used herein, “subject” refers to an animal, such as a mammal (including a human), that has been or will be the object of treatment, observation or experiment. “Subject” and “patient” may be used interchangeably, unless otherwise indicated. Mammals include, but are not limited to, mice, rodents, rats, simians, humans, farm animals, dogs, cats, sport animals, and pets. The methods described herein may be useful in human therapy and/or veterinary applications. In some embodiments, the subject is a mammal. In some embodiments, the subject is a human.

The term “dental surface” refers to a surface of a tooth made of hard tissue that can be treated with the peptides of the present technology. The hard tissue may include enamel, dentin, and/or cementum.

Unless defined otherwise, all technical and scientific terms used herein have the same meaning as commonly understood by one of ordinary skill in the art to which this present technology belongs. Although any methods and materials similar or equivalent to those described herein can also be used in the practice or testing of the present technology, representative illustrative methods and materials are described herein.

Throughout this disclosure, various publications, patents, and published patent specifications are referenced by an identifying citation. Also within this disclosure are Arabic numerals referring to referenced citations, the full bibliographic details of which are provided subsequent to the Examples section. The disclosures of these publications, patents, and published patent specifications are hereby incorporated by reference into the present disclosure to more fully describe the present technology.

### The Present Technology

There is a continuing need to improve treatment of dental caries. Polymeric restorative materials have revolutionized the treatment of dental caries by allowing clinicians a “one-of-a kind” in situ tissue engineering approach afforded by resin-dentin bonding [1-5]. Despite significant advances in composite resins for treating teeth, the low durability of the current dental adhesives continues to be a major health burden. The Global Oral Health Status 2022 report released by the World Health Organization (WHO) estimated 2 billion people worldwide suffer from caries in their permanent teeth [6,7]. The retention of composite resins relies on the adhesive system, which infiltrates into the collagen matrix to adhere to dentin [1,8-11]. Adhesion and adhesive performance on dentin are especially challenging due to the complexity of dentin, a mineralized dynamic biological composite tissue composed of 70% hydroxyapatite (HA), 30% type I collagen, multiple non-collagenous proteins, and water [12-14]. Due to the multi-scaled and multi-faceted events taking place at this complex resin-dentin adhesive interface, strategies developed so far have been slow to address the short lifespan of resin-dentin bonding and subsequently prevent the repeated failure of composite resin restorations [3,15]. Composite restorations will substantially benefit from the next generation of approaches based upon bioactive and biohybrid pathways that address the ongoing multifaceted events at this complex interface [2,15-18].

Resin-dentin bonding can be facilitated through acid-etching to remove the mineral phase which provides space to facilitate the infiltration of the adhesive system into the collagen matrix of dentin. Structurally homogeneous infiltration of the resin monomers into this complex matrix is difficult. Monomers are expected to fill the intra- and inter-fibrillar spaces within the collagen network and polymerize in situ to fully seal the region [4,10,19,20]. However, the resulting resin-dentin interdiffusion zone suffers from poor infiltration of resin monomers into the demineralized dentin matrix (collagen network). Penetration capacity of the monomers, caries pathophysiology, the expanded state of dentin collagen structure, and the variation on mineral content all affect the integrity of the resin-dentin interdiffusion zone. Unbound and entrapped water in the collagen network further accelerates the hydrolysis of resin and intensifies the vulnerability at this hybrid layer.

Despite the numerous approaches that have been developed to eliminate residual water from the collagen network, residual water is trapped within the hybrid layer. The residual water interferes with polymerization, plasticizes the adhesive and facilitates hydrolysis of the ester bonds in the methacrylate-based adhesives. The carboxylate and alcohol by-products of the ester hydrolysis are even more hydrophilic than the original adhesive network, thus increasing water sorption into the hybrid layer causing a cascade of degradation [4,8,10,11,21-24]. This self-perpetuating feed-forward cycle is aggravated by the insufficient infiltration of adhesive into the demineralized dentin collagen [25,26]. Consequently, the water-rich, highly porous collagen fibrils become exposed, allowing further sorption into the adhesive. Over time, degradation reaches a point where microleakage starts to occur, expediating the diffusion of oral fluids (containing e.g., salivary esterase) and bacteria (e.g., S. mutans) into the site [4,19,22,24,26-28]. These contaminants accelerate the degradation cascade of the adhesive, thus compromising its bond integrity, furthering tooth demineralization and caries progression at the site-resulting in the ultimate failure of the restoration [4,20-22,24]. It is recognized that the unprotected collagen fibers have a highly porous structure. Strategies that enable achieving better penetration and protecting the collagen network would be key to providing interfacial stability at the complex dentin/adhesive interface.

The dentin extracellular matrix contains collagens, non-collagenous proteins, and self-assembled collagen fibrils cooperating to stabilize and guide mineral growth. Collagen mineralization is considered to be mediated by interactions between negatively charged complexes of amorphous calcium phosphate (ACP) precursors and the collagen fibers. The ACP precursors are formed due to interactions between ionic components with the proteins controlling the mineral deposition and phase transformation precipitation. The ACP precursors penetrate the collagen fibrillar matrix and then transform into hydroxyapatite, resulting in the excellent mechanical properties observed in mineralized dentin. Developing a better understanding of the biomineralization processes and mechanisms has accelerated biomimetic design strategies for tissue repairs [15,29]. Non-collagenous proteins, composed mainly of acidic residues and their analogs, as well as peptides enriched in acidic amino acids were proposed to stabilize the mineralization and prevent degradation at the soft-hard tissue interfaces including the dentin/adhesive interface [30-33].

Intrafibrillar remineralization of collagen at the “hybrid layer” could be a critical pathway to address the vulnerability of the exposed demineralized dentin collagen and improve long-term repair of the tooth [36]. Recent approaches for the intrafibrillar remineralization with peptides involve using the casein phosphopeptide-amorphous calcium phosphate complex [37,38], beta-sheet self-assembled peptide hydrogels (P11-4 [39], ID8 [40], RAD/KLT [41]), amelogenin-derived peptide P26 [42], and mineralization-promoting peptides (MMP3 [43,44]) among others. These methods either focus on mineral formation at the site or involve stabilizing the collagen fibrils by generating a scaffold to achieve intrafibrillar mineralization [45]. While all these approaches could be promising, none of these approaches are specifically targeting collagen accompanied by mineral deposition taking place at collagen intrafibrillar and interfibrillar sites.

The present technology described herein addresses these needs and vacancies in the art.

### Bifunctional Peptides

In an aspect, the present technology provides a bifunctional peptide of amino acid sequence TKKLTLRT-X₁-MLPHHGA (SEQ ID NO: 1) or one or both of a pharmaceutically acceptable salt thereof and a solvate thereof, where X₁ is absent (a bond) or is a spacer of 1, 2, 3, 4, 5, 6, 7, 8, 9, or 10 amino acids. The bifunctional peptide of the present technology is also alternatively referred to herein as “a peptide of the present technology,” “the peptide of the present technology,” “the peptide,” and the like. The bifunctional peptide of the present technology may include one or more D-amino acids as well as one or more L-amino acids. In any embodiment herein, the bifunctional peptide may consist of only D-amino acids, or alternatively in any embodiment herein the bifunctional peptide may consist only of L-amino acids. As discussed herein, the bifunctional peptide of the present technology includes a collagen-binding peptide portion, an optional spacer, and a hydroxyapatite binding peptide portion. The bifunctional peptide of the present technology achieves 1) targeted surface binding to dental surfaces, 2) collagen infiltration, and/or 3) the growth of new calcium phosphate mineral layers on the dental surface.

A bifunctional peptide of the present technology may be synthesized by any technique known to those of skill in the art and by methods as disclosed herein. Methods for synthesizing the disclosed peptides may include chemical synthesis of proteins or peptides, the expression of peptides through standard molecular biological techniques, and/or the isolation of proteins or peptides from natural sources. The disclosed peptides thus synthesized may be subject to further chemical and/or enzymatic modification. Various methods for commercial preparations of peptides and polypeptides are known to those of skill in the art.

A bifunctional peptide of the present technology may alternatively be made by recombinant means or by cleavage from a longer polypeptide. The composition of a peptide may be confirmed by amino acid analysis or sequencing.

As discussed above, the bifunctional peptide may or may not include a spacer of 1, 2, 3, 4, 5, 6, 7, 8, 9, or 10 amino acid residues. In any embodiment including a spacer, X₁ may be EAAAK (SEQ ID NO: 2), APA, GGG, PAPAP (SEQ ID NO: 5), GSGGG (SEQ ID NO: 6), KGSVLSA (SEQ ID NO: 7), PKSALQEL (SEQ ID NO: 8), GLALLGWG (SEQ ID NO: 9), LGWLSAV (SEQ ID NO: 10), WLMNYFWPL (SEQ ID NO: 11), or YLMNYLLPY (SEQ ID NO: 12).

In any embodiment herein, the bifunctional peptides according to the present technology may be

(SEQ ID NO: 13)
TKKLTLRTEAAAKMLPHHGA,
(SEQ ID NO: 14)
TKKLTLRTAPAMLPHHGA,
(SEQ ID NO: 15)
TKKLTLRTGGGMLPHHGA,
(SEQ ID NO: 16)
TKKLTLRTPAPAPMLPHHGA,
(SEQ ID NO: 17)
TKKLTLRTGSGGGMLPHHGA,
(SEQ ID NO: 18)
TKKLTLRTKGSVLSAMLPHHGA,
(SEQ ID NO: 19)
TKKLTLRTPKSALQELMLPHHGA,
(SEQ ID NO: 20)
TKKLTLRTGLALLGWGMLPHHGA,
(SEQ ID NO: 21)
TKKLTLRTLGWLSAVMLPHHGA,
(SEQ ID NO: 22)
TKKLTLRTWLMNYFWPLMLPHHGA,
(SEQ ID NO: 23)
TKKLTLRTYLMNYLLPYMLPHHGA,
or
(SEQ ID NO: 24)
TKKLTLRTMLPHHGA.

In addition, in any embodiment herein the bifunctional peptides may mineralize and/or remineralize hard tissue. Specifically, the bifunctional peptides may facilitate tissue repair and restoration by depositing calcium phosphate as hydroxyapatite, and/or may biomineralize enamel by regulating and initiation the growth of calcium phosphate (e.g., hydroxyapatite) mineral. In this way, the bifunctional peptides direct remineralization of defective enamel and/or dentin.

A wide range of distinct calcium phosphate phases exist in mineralized tissues and these phases are commonly classified by the Ca/P molar ratio. Biomineralized tissue formed in the presence of a bifunctional peptide of the present technology may have an average Ca/P ratio of about 1.20 to about 1.67. Thus, in any embodiment herein, biomineralized tissue formed in the presence of a bifunctional peptide of the present technology may have an average Ca/P ratio of about 1.20, about 1.25, about 1.30, about 1.35, about 1.40, about 1.45, about 1.50, about 1.55, about 1.60, about 1.61, about 1.62, about 1.63, about 1.64, about 1.65, about 1.66, about 1.67, or any range including and/or in between any two of these values. For example, the Ca/P ratio of the biomineralized tissue may have a Ca/P ratio of about 1.35 to about 1.70, including about 1.63 for enamel and about 1.61 for dentine. The biomineralized tissue may have a Ca/P ratio consistent with octacalcium phosphate (having a Ca/P ratio of 1.33), amorphous calcium phosphate (having a Ca/P ratio of 1.50) or hydroxyapatite (having a Ca/P ratio of 1.67), or any value there between.

### Compositions

In an aspect, a composition is provided that includes a bifunctional peptide of any embodiment disclosed herein, a pharmaceutically acceptable carrier or one or more excipients, fillers or agents (collectively referred to hereafter as “pharmaceutically acceptable carrier” unless otherwise indicated and/or specified). In a related aspect, a medicament for controlling enhancing collagen infiltration from a dental surface to a dental composite is provided that includes a bifunctional peptide of any embodiment disclosed herein and optionally a pharmaceutically acceptable carrier. In a related aspect, a pharmaceutical composition is provided that includes an effective amount of a bifunctional peptide of any embodiment disclosed herein as well as a pharmaceutically acceptable carrier. For ease of reference, the compositions, medicaments, and pharmaceutical compositions of the present technology may collectively be referred to herein as “compositions.” In further related aspects, the present technology provides methods and uses that include a bifunctional peptide of any aspect or embodiment disclosed herein and/or a composition of any embodiment disclosed herein as well as uses thereof.

“Effective amount” refers to the amount of a bifunctional peptide or composition required to produce a desired effect. One example of an effective amount includes amounts or dosages that yield acceptable toxicity and bioavailability levels for therapeutic (pharmaceutical) use including, but not limited to, enhancing collagen infiltration from a dental surface to a dental composite, facilitating the formation of new calcium phosphate mineral layers on dental surfaces, improving adhesion between a dental surface and a dental composite, and/or rebuilding damaged dental tissue. In any aspect or embodiment disclosed herein (collectively referred to herein as “any embodiment herein,” “any embodiment disclosed herein,” or the like) of the compositions, pharmaceutical compositions, and methods including a bifunctional peptide of the present technology, the effective amount may be an amount effective in treatment, including, but not limited to, enhancing collagen infiltration from a dental surface to a dental composite, facilitating the formation of new calcium phosphate mineral layers on dental surfaces, improving adhesion between a dental surface and a dental composite, and/or rebuilding damaged dental tissue. By way of example, the effective amount of any embodiment herein including a bifunctional peptide of the present technology may be from about 0.01 μg to about 200 mg of the bifunctional peptide (such as from about 0.1 μg to about 50 mg of the bifunctional peptide or about 10 μg to about 20 mg of the peptide). The effective amount may be related to the corresponding area and the molecular mass of the bifunctional peptide required to saturate a dental surface. The molecular mass required to deliver the corresponding surface coverage could be obtained by converting the number of bifunctional peptides that is calculated from the theoretical “footprint” for each bifunctional peptide using the variety of peptide structural analyses tools including UCSF Chimera tool. See E. Cate Wisdom, Yan Zhou, Casey Chen, Candan Tamerler, and Malcolm L. Snead, Mitigation of Peri-implantitis by Rational Design of Bifunctional Peptides with Antimicrobial Properties, ACS Biomaterials Science & Engineering 2020 6 (5), 2682-2695 DOI: 10.1021/acsbiomaterials.9b01213. The “theoretical footprint” of the bifunctional peptides could be determined through the length and width distance values measured from the α-carbon of amino acid residues. The number of bifunctional peptides could be next converted to a molecular mass required to deliver the corresponding dental surface coverage. The methods and uses according to the present technology may include an effective amount of a bifunctional peptide of any embodiment disclosed herein. In any aspect or embodiment disclosed herein, the effective amount may be determined in relation to a subject and/or in relation to dental caries. The term “subject” and “patient” may be used interchangeably.

Thus, the instant present technology provides pharmaceutical compositions and medicaments including a bifunctional peptide of any embodiment disclosed herein (or a composition of any embodiment disclosed herein) and a pharmaceutically acceptable carrier. The compositions may be used in the methods and treatments described herein. The pharmaceutical composition may be packaged in unit dosage form. The unit dosage form is effective in treatment, including enhancing collagen infiltration from a dental surface to a dental composite, facilitating the formation of new calcium phosphate mineral layers on dental surfaces, improving adhesion between a dental surface and a dental composite, and/or rebuilding damaged dental tissue. Generally, a unit dosage including a bifunctional peptide of the present technology will vary depending on patient considerations. Such considerations include, for example, age, protocol, condition, sex, extent of disease, contraindications, concomitant therapies and the like. Further, a unit dosage including a bifunctional peptide of the present technology may vary depending on the size of the dental surface to be treated. An exemplary unit dosage based on these considerations may also be adjusted or modified by a physician skilled in the art. Suitable unit dosage forms, include, but are not limited to oral solutions, powders, lozenges, topical varnishes, lipid complexes, liquids, etc.

The pharmaceutical compositions and medicaments may be prepared by mixing a bifunctional peptide of the present technology with one or more pharmaceutically acceptable carriers, excipients, binders, diluents or the like. Such compositions may be in the form of, for example, powders, syrup, emulsions, suspensions, or solutions. The instant compositions may be formulated for various routes of administration, for example, by intraoral administration or via administration (e.g., application) to a dental surface external to a patient. The following dosage forms are given by way of example and should not be construed as limiting the instant present technology.

A bifunctional peptide of the present technology may be incorporated into adhesive formulations, such as impregnated into the adhesive. The adhesive may be applied to the exposed tooth surface. The dental composite, which is placed on top of the adhesive, forms a bond with the adhesive. Thus, in an aspect, the present technology provides a dental adhesive composition that includes a bifunctional peptide of any disclosed herein and one or more dental adhesives. The dental adhesive composition may include an effective amount of the bifunctional peptide for treating a dental surface. In any embodiment herein, the bifunctional peptide is present at a concentration of about 5 μM to about 500 μM (including about 5 μM, about 10 μM, about 20 μM, about 50 μM, about 100 μM, about 150 μM, about 200 μM, about 500 μM, or any range including and/or in between any two of these values).

For intraoral administration, powders and suspensions are acceptable as solid dosage forms. These may be prepared, for example, by mixing a bifunctional peptide of the instant present technology with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms may contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents and/or perfuming agents.

Liquid dosage forms for oral administration (e.g., intraoral administration) may be in the form of pharmaceutically acceptable emulsions, syrups, suspensions, or solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral administration.

As noted above, suspensions may include oils. Such oils include, but are not limited to, peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as, but not limited to, ethanol, isopropyl alcohol, hexadecyl alcohol, glycerol, and propylene glycol. Ethers, such as but not limited to, poly(ethyleneglycol), petroleum hydrocarbons such as mineral oil and petrolatum; and water may also be used in suspension formulations.

The pharmaceutical compositions and medicaments in liquid or gel form may have a concentration of a bifunctional peptide of the present technology sufficient to provide an effective amount as described above. The concentration of the bifunctional peptide of the present technology in the pharmaceutical compositions and medicaments may be about 5 M to about 500 μM (including about 5 μM, about 10 μM, about 20 μM, about 50 μM, about 100 μM, about 150 μM, about 200 μM, about 500 μM, or any range including and/or in between any two of these values).

The pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. The formulations may optionally contain stabilizers, antimicrobial agents, antioxidants, pH modifiers, surfactants, bioavailability modifiers, and combinations of these. The carriers and stabilizers vary with the requirements of the particular composition, but typically include nonionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Powders and sprays may be prepared, for example, with excipients such as lactose, talc, silicic acid, aluminum hydroxide, calcium silicates and polyamide powder, or mixtures of these substances. Ointments, pastes, creams, and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), and “Remington: The Science and Practice of Pharmacy,” 20th Edition, Editor: Alfonso R Gennaro, Lippincott, Williams & Wilkins, Baltimore (2000), each of which is incorporated herein by reference.

### Methods

In an aspect, a method of adhering a dental composite to a dental surface is provided where the method includes administering to the dental surface a bifunctional peptide of any embodiment disclosed herein, administering to the dental surface a composition of any embodiment disclosed herein, and/or administering to the dental surface a dental adhesive composition of any embodiment disclosed herein, to provide a treated dental surface; and contacting the dental composite with the treated dental surface. The method may, among other things, facilitate the formation of new calcium phosphate mineral layers on dental surface and/or improve adhesion by facilitating collagen infiltration.

In any embodiment disclosed herein of the method, the administering step may include administering an effective amount of the bifunctional peptide for treating the dental surface. In any embodiment disclosed herein, the dental surface may be a dental enamel and/or dentin, where the dental enamel and/or dentin may include a carious region, a hypomineralized region, or both a carious region and a hypomineralized region.

In any embodiment disclosed herein of the method, the administering step may include contacting the dental surface with the bifunctional peptide, the composition, and/or the dental adhesive composition for a period of about 10 seconds to about 4 hours, such as for a period of about 10 seconds to about 20 seconds, about 20 seconds to about 1 minute, about 1 minute to about 4 hours, 1 minute to 10 minutes, or 2 hours to 4 hours.

The examples herein are provided to illustrate advantages of the present technology and to further assist a person of ordinary skill in the art with preparing or using the bifunctional peptides and compositions of the present technology. The examples herein are also presented in order to more fully illustrate the preferred aspects of the present technology. The examples should in no way be construed as limiting the scope of the present technology, as defined by the appended claims. The examples may include or incorporate any of the variations, aspects, or embodiments of the present technology described above. The variations, aspects, or embodiments described above may also further each include or incorporate the variations of any or all other variations, aspects, or embodiments of the present technology.

### EXAMPLES

### Materials and Methods

### Materials

Peptide synthesis required N-methyl morpholine (NMM), Wang amide resin, Fmoc-resin, Fmoc-amino acid building blocks, D-biotin, piperidine, and 2-(1H-benzotriazole-1-yl)-1,1,3,3-tetramethyluranium hexafluorophosphate (HBTU), which were purchased from AAPPTec LLC (Louisville, KY, USA). N, N-Dimethylformamide (DMF, 99.8%), trifluoroacetic acid (TFA, 99%), triisopropylsilane (98%), thioanisole (99%), and diethyl ether (99%) were obtained from Sigma-Aldrich (St. Louis, MO, USA). 1,2-ethanedithiol (95%), N,N Diisopropylethylamine (99.5%, nitrogen flushed), and hydroxymethyl (Tris) aminomethane hydrochloride (Tris-HCl, 99%+, extra pure) were purchased from Acros Organics (NJ, USA). Phenol (89%), calcium chloride dihydrate (99.7%), and sodium hydroxide (97%) were obtained from Fisher Scientific (Fair Lawn, NJ, USA). Glycerophosphate calcium salt was purchased from MP Biomedicals LLC (Solon, OH, USA) and 6N Hydrochloric acid solution from Fisher Chemical (Fair Lawn, NJ, USA). All chemicals in this study were used without further purification. We acquired FastAP Thermosensitive Alkaline Phosphatase from Thermo Fisher Scientific (Vilnius, Lithuania). Collagen Type 1 solution from rat tail (3 mg/mL, C3867, 95%) was purchased from Sigma-Aldrich (St. Louis, MO, USA). Highest grade V1 12 mm Mica discs and 12 mm atomic force microscopy specimen discs were obtained from Ted Pella Inc. (Redding, CA, USA). STKYDOT, an atomic force microscopy specimen disc adhesive, was purchased from Bruker Corporation (Camarillo, CA, USA).

### Peptide Design

The selection of the collagenase-collagen-binding peptide (CBP) sequence, TKKLTLRT (SEQ ID NO. 25), was based as an analog of the original peptide sequence, TKKTLRT (SEQ ID NO: 27), reported by de Souza et al. (1992) [58]. The latter corresponds to the amino acid sequence derived from the nucleotide sequence of the complementary DNA strand coding for the human pro-α2(I) collagen domain, a molecule attacked by human fibroblast collagenase. de Souza et al. used the principle of hydropathic complementarity to develop a peptide sequence that would result in sense collagen domain to antisense peptide sequence binding given respective hydrophobic-hydrophilic residue interactions [69-71]. Employing that same principle, analogs to TKKTLRT (SEQ ID NO: 27) were explored to identify hydropathic profiles that showed better alignment with respect to the amino acid sequence of the sense strand of the gene. From this, the sequence TKKLTLRT (SEQ ID NO: 25) was chosen to be implemented. In addressing the biomineralization prong of this study, the hydroxyapatite-binding peptide (HABP1) sequence MLPHHGA (SEQ ID NO. 26) was selected. The sequence was developed by Gungormus et al. (2008) [34] where it showed the highest binding affinity in directing the mineralization process of calcium phosphate (Ca—P). An exemplary bifunctional peptide bifunctional peptide of the present technology was generated by combining these the CBP and HABP1 motifs but separated by a short and rigid spacer: TKKLTLRT-APA-MLPHHGA (SEQ ID NO. 14; “CBP-HABP1”).

### Peptide Synthesis

The CBP, HABP1, and CBP-HABP1 peptides were synthesized on the AAPPTEC Focus XC synthesizer (AAPPTec, Louisville, KY, USA), using a standard Fmoc solid-phase peptide synthesis protocol. The peptides were synthesized on Wang resin with the subsequent resin-bound peptides cleaved and their side chains deprotected, resulting in the canonical C-terminus functional group of carboxylic acid. The cleavage cocktail of CBP contains the following: TFA, phenol, triisopropylsilane, and water (90:5:2.5:2.5, v/v percent). For HABP1, the cleavage cocktail is as follows: TFA, thioanisole, ethanedithiol, triisopropylsilane, and water (87.5:5.0:2.5:2.5:2.5, v/v percent). Lastly, the cleavage cocktail for the bifunctional peptide CBP-HABP1 contains TFA, phenol, thioanisole, water, ethanedithiol, and triisopropylsilane (81.5:5.0:5.0:5.0:2.5:1.0, v/v percent). The peptides were left in cleavage cocktail on a rotator for 2 h, precipitated in cold ether, and then lyophilized.

Crude peptide purification was performed on a semi-preparative reversed-phase high pressure liquid chromatography (HPLC) Waters system, containing a Waters 600 controller and Waters 2487 Dual Absorbance Detector, using a 10 μm C-18 silica Luna column (250× 10 mm, Phenomenex Inc., Torrance, CA, USA). The mobile phase is composed of phase A (94.5% HPLC grade water, 5% acetonitrile, 0.1% TFA) and phase B (100% acetonitrile). Lyophilized peptides were dissolved in 4 mL of phase A and purified at 0.5% phase B*×min⁻¹ on a linear gradient (5-85% phase B), performed at 3 mL*×min⁻¹, room temperature, with detection at 254 nm. Purified fractions collected were verified by the analytical Shimadzu HPLC system, composed of an LC-2010 HT liquid chromatograph and SPD-M20A prominence diode array detector, with a 5 μm C-18 silica Luna column (250×4.6 mm, Phenomenex Inc., CA, USA). This mobile phase is composed of phase A (99.9% HPLC-grade water, 0.1% TFA) and phase B (100% acetonitrile) with the system run on a linear gradient with 1 mL*×min⁻¹ flow, 40° C., detection at 254 nm. The purified peptides were lyophilized and stored at −20° C.

### Fourier Transform-Infrared Spectroscopy (FT-IR)

The molecular structures of peptides were verified using the PerkinElmer Frontier IR spectrometer with the universal attenuated total reflectance polarization accessory (Waltham, MA, USA). Lyophilized peptide was evaluated across the wavelength range of 4000 cm⁻¹-550 cm⁻¹ at a 4 cm⁻¹ spectral resolution.

### Circular Dichroism Spectroscopy (CD)

The secondary structure of each of the three peptides, CBP, HABP1, and CBP-HABP1 were measured through circular dichroism (CD) spectra. Measurements were made with CD spectrometer (JASCO, J-815) at room temperature, using a 1.0 mm cuvette. Each peptide sample was dissolved at 0.2 mg/mL in 10 mM potassium phosphate (pH 7.4) at 4° C. for 16 h. Spectra shown are averaged from three experimental repeats. The scans were acquired from 190 to 300 nm at a scanning speed of 60 nm/min. CD spectra were processed for secondary structure composition with the tools of CD Pro. For each peptide, the reference set selected was SMP50. The mean residue ellipticity (MRE) was analyzed with CD Pro software to compare likely conserved secondary structure features from the single domains (CBP and HABP1) in the chimeric peptide (CBP-HABP1) [51]. Absorbance measurements were taken every 1 nm as the average of 5 technical replicates and smoothed by a 7-point Savitzky-Golay filter. The fractions of secondary structure (Regular Helix, Distorted Helix, Regular Sheet, Distorted Sheet, Turns, and Unordered) were averaged for all three CD Pro tools (SELCON3, CDSSTR, and CONTILL).

### Collagen-Peptide Sample Preparation

Grade V1 round mica, 0.21 mm thick and 12 mm in diameter, was used as the substrate surface and mounted onto a 12 mm atomic force microscopy specimen disc using STKYDOT adhesive. In 1 mL of 3 mg/mL Type 1 rat tail collagen suspension, 50 nanomoles of peptide were added. The ratio of mass collagen to mass peptide ranged from 100 wt collagen: 1 wt peptide for single domains to 100 wt collagen: 3 wt peptide for the chimeric peptide. Once peptide was dissolved and gently mixed in the collagen suspension, 150 μL was drop-cast onto the substrate surface and left to self-assemble at 4° C. for 16 h. The preparation resulted in mica surfaces with a coating layer of collagen-peptide, observed as “dry” by visual inspection.

### ALP-Mediated Mineralization

ALP-driven mineralization was executed following the protocol discussed in Gungormus et al. (2008) [34]. The mineralization buffer prepared was composed of 24.4 Ca²⁺ mM and 14.4 mM B-glycerophosphate made in 25 mM Tris-HCl buffer at pH 7.4, and 200 μL/well was added to a 24-well plate. Collagen-peptide samples were placed gently within the well to lay flat at the bottom. The mineralization reaction was initiated by adding FastAP (thermosensitive alkaline phosphatase), 1.4×10⁻⁶ g/mL, and left to incubate at 37° C. for 20 min. The samples were then removed from the wells, gently rinsed in sterile-filtered water, and left to air dry overnight. Throughout this process the samples were handled in a horizontally flat manner to prevent disturbing the peptide-functionalized collagen layer on the surface of the substrate.

### RAMAN Spectroscopy

Demineralized dentin samples, adhesive/dentin samples (pre/post mineralization), and SC collagen on glass were imaged using a LabRAM ARAMIS Raman microscope (HORIBA Jobin Yvon, Edison, NJ, USA), equipped with a HeNe laser (2=663 nm, laser power=17 mW). Light micrograph images were taken using a 50× long working distance objective Olympus lens. The samples spectra were evaluated over a wavelength range of 300 cm⁻¹ to 1800 cm⁻¹ with a 15 s spectra acquisition time of 4 acquisitions per cycle and processed using LabSPEC 6 software (HORIBA Jobin Yvon, Edison, NJ, USA). Divisive Clustering Analysis (DCA) was used to classify and group respective spectra. This multivariate analysis method was integrated into the LabSPEC 6 analysis software as a Multivariate Analysis (MVA) module (powered by Eigenvector Research Inc., HORIBA Jobin Yvon, NJ, USA). A rectangular area of the surface was imaged and submitted to this multivariate analysis where a statistical pattern determined derived independent clusters to present chemically distinct regions [3,4]. Average spectra are calculated per cluster which were used to provide information on particular peak parameters and component distribution in the resulting spectra. These components were analyzed for relative degree of mineralization via mineral-to-matrix ratio (MMR), inferred from the ratio of peak intensities at 960 cm⁻¹ (phosphate) and 1460 cm⁻¹ (CH₂ bending), crystallinity via full width at half maximum (FWHM) from the v1 phosphate band (960 cm⁻¹), and the carbonate content of mineral crystallites via gradient in mineral content (GMC), based on ratio of relative carbonate (10⁷⁰ cm⁻¹) and phosphate (960 cm⁻¹) peak heights. The calcium-phosphate (Ca/P) to collagen ratio was also assessed to understand the relative thickness of the mineral formed during mineralization [3]. This would help in comparative evaluations between the samples.

### PeakForce-QNM AFM Imaging

A Bruker Multimode 8 HR scanning probe microscope (Bruker Nano Inc., Camarillo, CA, USA) was operated in peak force tapping mode with the capability of Quantitative Nanomechanics (PeakForce-QNM) in air mode conditions (24±2° C., 40%±5% RH). This advanced testing mode was used to examine the topographical and nanomechanical property changes of the collagen-peptide samples both before and after mineralization. Tapping mode etched silicon probes, type RTESPA 525-30 (Bruker Nano Inc., Camarillo, CA, USA) with a resonant frequency of about 518 kHz, were used to acquire images (1 μm×3 μm and 5 μm×5 μm) at scan rate of 0.5 Hz with 512 pixel/line resolution. The images of the samples were recorded using NanoScope 8.15 software and analyzed using NanoScope Analysis 2.0 software (Bruker Nano Inc., Camarillo, CA, USA).

### SEM/EDX Imaging of Mineralized Collagen-Peptide Samples

In order to examine initial intrafibrillar mineral formation and morphology and determine Ca/P ratios precipitated on the peptide-functionalized collagen surface, a Cold Field Emission Scanning Confocal Microscope (SEM, S-4700 model, Hitachi High-Tech America, Schaumburg, IL, USA) equipped with a silicon drift energy-dispersive detector (EDS, X-max, Oxford Instruments, Concord, MA, USA) was used via the Microscopy and Analytical Imaging Research Resource Core Laboratory (RRID:SCR_021801). Collagen-peptide samples were sputter-coated with 3 nm gold using a Quorum sputter coating system (Q150, Quorum, Laughton, East Sussex, UK). SEM imaging was completed at an acceleration voltage of 10 kV at ultra-high resolution operating mode and EDS measurements were made at 10 kV under normal operating mode to preserve the integrity of the collagen-peptide layer and prevent burning during signal capture. EDS analysis was performed through AZtec software (X-MaxN, Oxford Instruments, Concord, MA, USA).

### SEM Surface Mineral Mapping

SEM images were analyzed to examine the Ca—P mineral deposition changes between the different peptide-functionalized collagen platforms and to understand the relative amount of initial Ca—P mineral formed on those surfaces. The SEM images were processed through written code using Python 3.9.13 software. The tif files were preprocessed by median blurring and histogram equalization. Multi-Otsu threshold determination algorithm was applied, resulting in a binary matrix, with 1 corresponding to the location of mineral on the surface and 0 corresponding to a location of surface matrix (i.e. no mineral). The area of that represents the surface covered by mineral. The resulting binary image represents the surface mineral area, and the percent-area is the proportion of mineral surface locations to all locations in the image.

### MicroXCT Imaging

The microscale structure of the mineral formed within the peptide-incorporated collagen platform as a physical indicator of intrafibrillar mineralization, was observed using 3D X-ray microcomputed tomography (MicroXCT-400, Xradia Inc., Pleasanton, CA, USA). The transmission X-ray images of the samples were obtained using a tungsten anode setting of 50 kV at 8 W. A total of 1600 images were acquired at a resolution time of 15 s per image. The 3D images were reconstructed using the XM Reconstructor 8.0 software and were analyzed from an orthogonal view using a TXM 3D Viewer under the spatial surface display (SSD) mode (Xradia Inc., Pleasanton, CA, USA).

### Results

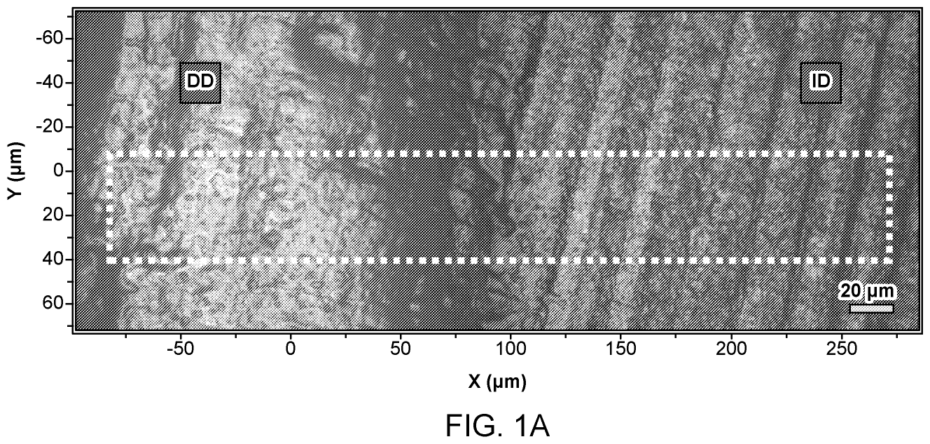

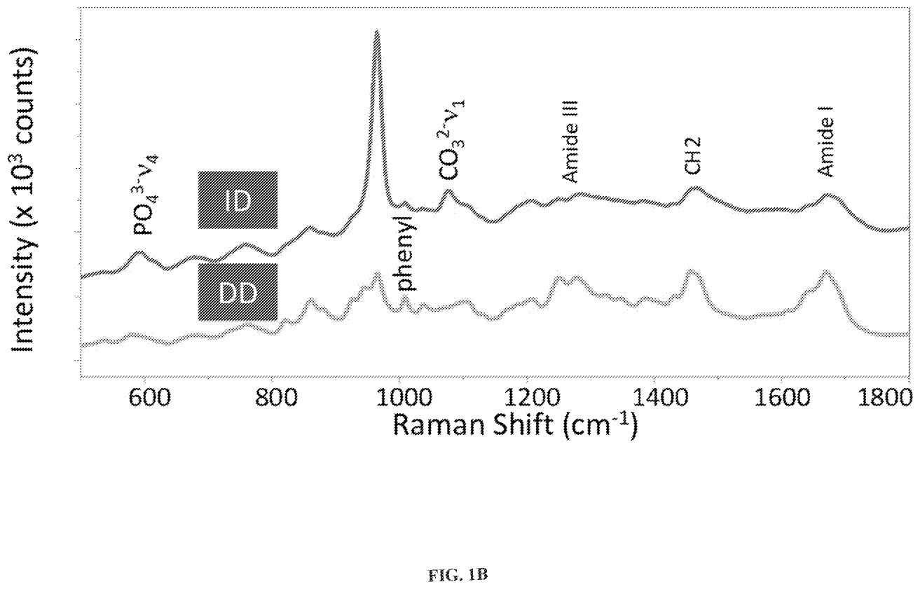

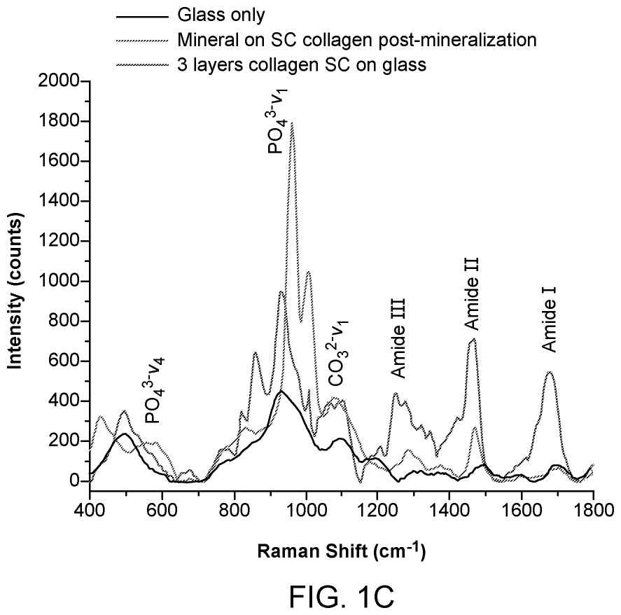

Human dentin and rat tail type I collagen samples were investigated as part of the initial studies. Two-dimensional RAMAN light microscopy was used to identify the differences in chemical signature between demineralized and intact dentin on a human tooth specimen. FIG. 1A provides the light micrograph representation of the two regions (“DD”=demineralized dentin; “ID”=intact dentin), with FIG. 1B reflecting the spectral analysis. For intact dentin, the characteristic peaks associated with PO₄³⁻ and CO₃²⁻ are observed at 960 cm⁻¹ and 10⁷⁰ cm⁻¹, respectively. The intensity of these mineral bands was decreased in the demineralized dentin, whereas the spectral signatures reflecting type I collagen became clearer: 1667 cm⁻¹ (amide I), 1460 cm⁻¹ (CH₂), and the doublet observed from 1215 cm⁻¹ to 1310 cm⁻¹ (amide III) [3,46,47]. The chemical signatures observed in both intact and demineralized dentin were compared to spin-coated (SC) rat tail type 1 collagen on glass using RAMAN microscopy. To mimic biological systems, we adapted alkaline phosphatase (ALP)-mediated mineralization. As a key enzyme, ALP promotes mineralization by hydrolyzing the pyrophosphate and releasing inorganic phosphate. Raman spectra of the collagen samples were obtained with and without undergoing alkaline phosphatase-mediated mineralization (FIG. 1C). The indicative collagen peaks observed in demineralized dentin and the mineral peaks seen with intact dentin were respectively observed in the SC collagen and the mineralized SC collagen. The amide peaks observed on the SC collagen samples were diminished in the mineralized SC collagen sample; however, the 1454-1460 cm⁻¹ Raman spectral feature associated with CH₂ related to collagen is notable.

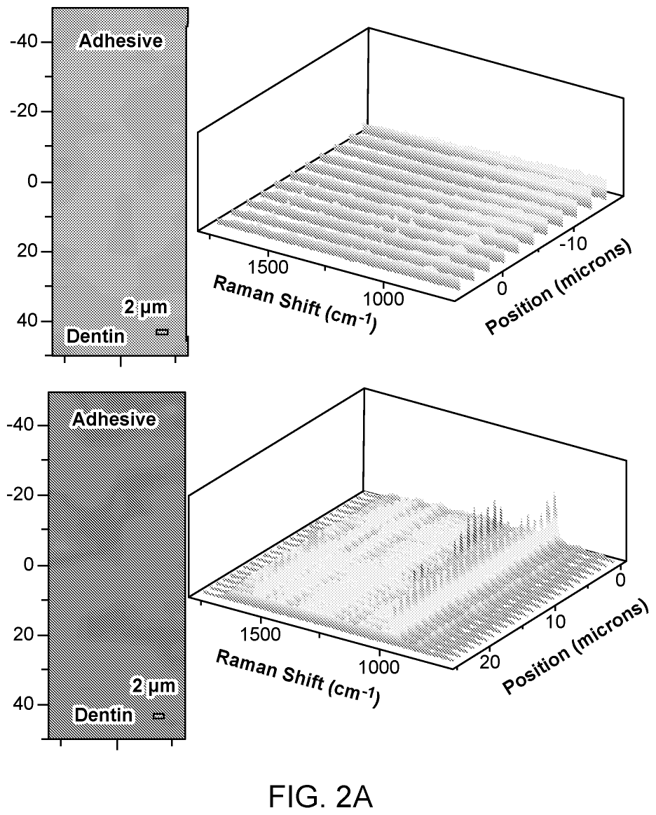

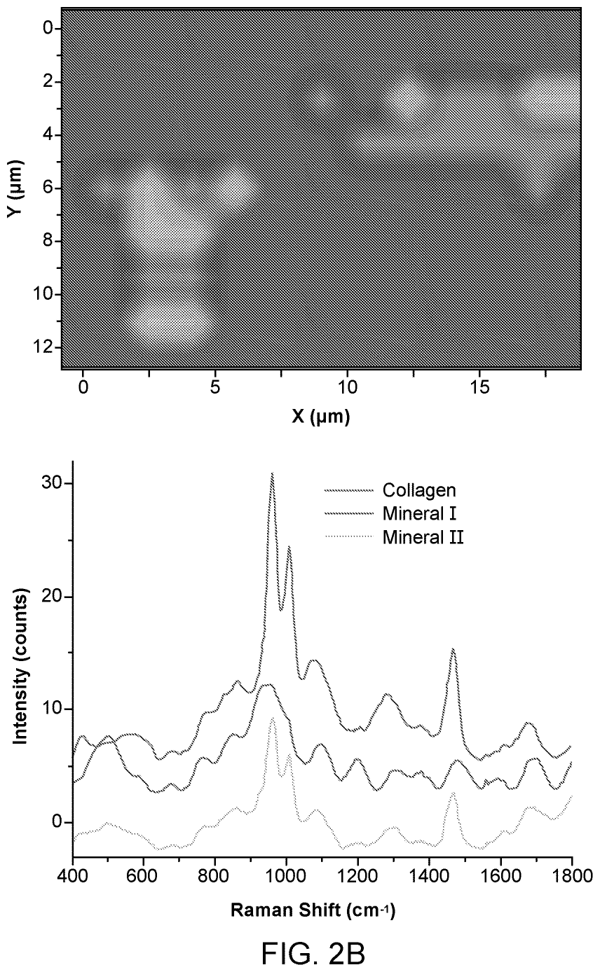

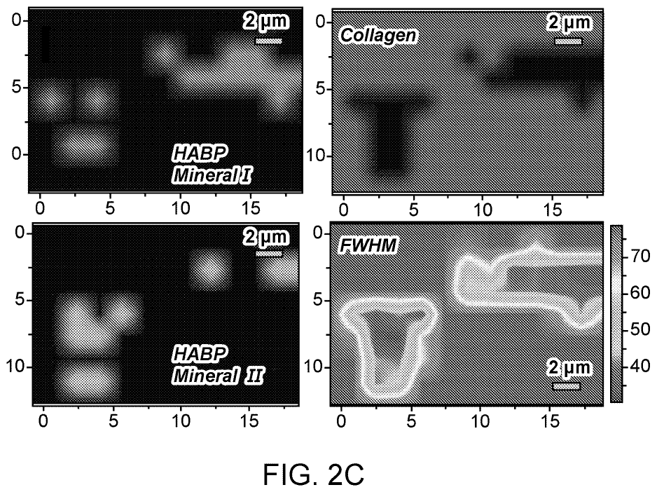

Next evaluated was the use of HABP1 to potentially direct mineralization within the adhesive/demineralized dentin hybrid layer. To do this RAMAN Divisive Clustering Analysis (DCA) was used, a hierarchical k-means clustering method which is useful for identifying the internal structure of multi-layer datasets. We mapped the most distinctive mineral composition variations associated with the collagen matrix structural variations according to their RAMAN spectra. Our mapping includes two groups, where HABP1 mineral group II shows a more mature mineral formed compared to that observed for HABP1 mineral group I, a distinction based on the narrower full-width half-maximum (FWHM) of the phosphate peak of mineral group I. FIG. 2A shows 2D RAMAN spectroscopy with and without HABP1 incorporation into the adhesive/dentin specimen, following the mineralization procedure. FIG. 2A shows that HABP1 mediates mineralization shown with a RAMAN peak consistent with CaPO₄ formation (960 cm⁻¹) which does not appear after the mineralization procedure without peptide. FIG. 2B shows the results of further analysis of the 2D Raman spectra, with pseudo-coloring being applied by the DCA method. The DCA method measures similarity in three parameters: ratio of 960/1460 peak (e.g., mineral-to-matrix ratio), ratio of 1078/960 (gradient mineral carbonation, GMC: carbonate to phosphate) peak, and ratio of the 960/1667 peaks (phosphate to collagen with retained amide I structure, Ca/P collagen). The spectral feature at 1460 cm⁻¹ is assigned to the CH₂ wag for collagen, and 1667 cm⁻¹ is the amide I peak for the collagen used [48]. The 960 cm⁻¹ peak is the v1 phosphate stretching vibration associated with the mineral and 10⁷⁸ cm⁻¹ is the band for v1 carbonate. The method clusters the following groups: collagen only, mineral group I, and mineral group II. This analysis method is summative, meaning that the mineral groups discovered are the mineral formation groups which are most distinctive. The distinction between the mineral groups is based on how much collagen is present at the location of the mineral, how much of the present collagen's two noted amide peaks signature are maintained, and by how much carbonate is in the mineral.

As shown in Table 1 below—where RAMAN spectral features are evaluated over HABP1 mineral types observed—mineral group I exhibits greater relative intensity of mineral to amide I (phosphate, 960)/amide I, 1667), an increased ratio of carbonate to phosphate (1078/960), and a decreased mineral-to-matrix ratio (phosphate/CH₂ wag, collagen). The value of 1667 cm⁻¹ has previously been noted as a beta-sheet structure [48]. Mineral group II exhibits less carbonate compared to mineral group I. Due to the lower carbonate content observed and narrower FWHM, i.e., full width at half maximum, we propose that mineral group II is a more mature mineral formation, being closer to hydroxyapatite compared to mineral group I.

TABLE 1

| | FMHW | MNRₚ | GMC | Ca-P/collagen |
|---|---|---|---|---|
| DCA Class | PO₄⁻³) | 960/1460 | 1078/960 | 960/1667 |
| HABP1 | 39.6 | 2.88 | 0.31 | 23.9 |
| mineral I | | | | |
| HABP1 | 35.6 | 3.59 | 0.22 | 13.2 |
| Mineral II | | | | |

FIG. 2C shows where the two mineral groups, which are represented by the spectra on the left, appear in the isolated DCA maps. The top right panel shows the DCA map dominated by collagen, the top left panel shows the DCA map of mineral group 1, and the bottom left panel shows the DCA map of mineral group II. Comparing the class spectra to the two mineral groups on the left, we see that the mineral groups are not segregated exclusively by how much mineral absorbance occurs at a location. This shows that one can distinguish the activity of HABP1 with respect to the background collagen and produce mature biomimetic mineral. When co-assembled with collagen, it is plausible to expect this peptide to direct biomimetic intrafibrillar mineralization at this complex interface.

To extend the remineralization capability into the exposed collagen network along the demineralized dentin matrix, a bifunctional chimeric peptide sequence was designed combining collagen-binding and remineralization domains using a spacer sequence. Using PEP-FOLD3, folded structures of the chimeric peptide sequence were compared with the corresponding folded domains alone, where the assumption was that identifying structural change in the incorporated chimeric peptide domain relates to change in the function of the domain. The extent of these changes was explored through the comparisons of different spacers with varying flexibility and length.

The spacers fit into three general categories according to the inter-domain relationships: (1) inter-residue contacts, (2) globular conformation, and (3) bi-function accessibility conformation. The inter-residue contacts limit the conformational flexibility of the domains the most directly, while the globular conformation limits the accessibility of the domain surfaces through shared interfaces by those domains. Investigated were helical motifs with low GRAVY scores—e.g., KGSVLSA (SEQ ID NO: 7), PKSALQEL (SEQ ID NO: 8)—and high GRAVY scores—e.g., GLALLGWG (SEQ ID NO: 9), LGWLSAV (SEQ ID NO: 10), WLMNYFWPL (SEQ ID NO: 11), and YLMNYLLPY (SEQ ID NO: 12). The limitation of these helical motifs was to impose conformation where the active domains shared interfaces. The most promising conformation type for retaining independent functions is the bi-functional accessibility conformation because each domain has the most access to interact with its molecular recognition partner. The GSGGG (SEQ ID NO: 6) was selected as short spacer sequence with high flexibility resulted in inter-domain contacts. The KGSVLSA (SEQ ID NO: 7) was selected due to its low flexibility to develop a shared domain interface. Conversely, the APA was selected as a very short sequence with low flexibility which offered the greatest accessibility for the domains, where this spacer was expected to reduce the probability of inter-domain interactions.

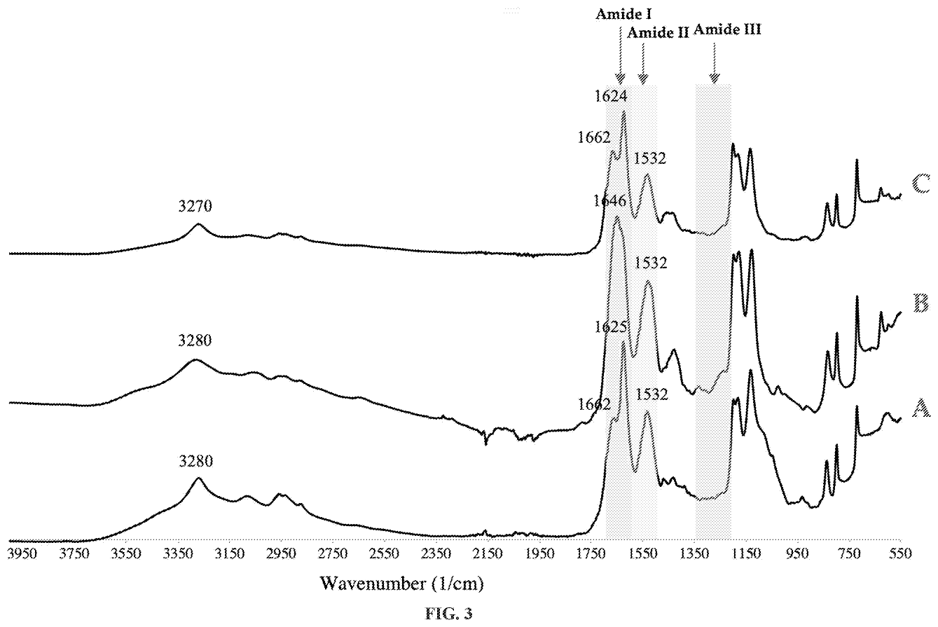

Based on the domain structure conservation approach analysis, we selected CBP-APA-HABP1 (i.e., TKKLTLRTAPAMLPHHGA (SEQ ID NO: 14), referred to hereafter alternatively as “CBP-HABP1”)) to initially test experimentally and the peptide synthesized. The secondary structures of the CBP, HABP1, and CBP-HABP1 were investigated using the FT-IR spectra in their lyophilized state, where FIG. 3 provides an summary of the results on overlaid spectra. The amide I band relates to the stretching of the backbone carbonyl. CBP-HABP1 and CBP have their highest amide I band peak related to beta-sheet structures with peak absorbance at 1624±1 cm⁻¹ [48]. CBP-HABP1 and CBP also share an amide I peak of 1662 cm⁻¹, which is related to α-helix formation. The dominant peak for lyophilized linear HABP1 is at 1646 cm⁻¹, which is in the random assignment region [48]. A main peak at 3280 cm⁻¹ was observed in the group A region (NH stretching) for both the CBP and HABP1 peptides, which was observed to be shifted to 3270 cm⁻¹ with CBP-HABP1. All peptides contained the same maximum in the amide II region (CN stretching and NH bending) at 1532 cm⁻¹ and lacked peaks in the amide III region (CN stretching and NH bending), as reflected in FIG. 3.

The in vitro structural changes of the peptides in solution were then investigated via circular dichroism spectroscopy (CD). The CDPro software package was used for CD spectral data processing and analysis [51]. The change in secondary structure composition of the chimeric peptide was compared to the mean secondary structure compositions of the single functional domains. The chimeric peptide appeared to develop a more unordered structure compared to the single functional domains. Still, the compositional changes for all the features were observed to be 10% or less.

### Effect of Peptide Incorporation on Collagen Self-Assembly

Building upon the conformational flexibility of the peptide, the CBP-HABP1 peptide was then studied for the assembly properties on the collagen fibrils on mica substrate using AFM topography image analysis. The images were acquired in tapping mode. Non-fibril features, indicated as intensity saturated regions on the surface, are acetic acid salt crystals from the evaporation of the solution in which the collagen was suspended. These features were excluded from image to prevent signal shadowing of the surface fibril assembly. Topographical analysis of the surfaces showed changes in the collagen fibril assembly with the incorporation of peptide in collagen prior to drop-casting. As shown in Table 2 below, collagen alone was observed to have an average fibril width of 98.89 nm, collagen+(CBP) showed a larger average fibril width of 121.4 nm, and collagen+ (CBP-HABP1) exhibited an average of 82.37 nm. However, with collagen+ (HABP1) the average fibril width rose to 129.9 nm, but the standard deviation more than tripled compared to that of the other samples (see Table 2). What was observed for the collagen+ (HABP1) was a mixture of larger fibrils as compared to fibrils formed with collagen alone or compared to fibrils formed with collagen+ (CBP) or collagen+ (CBP-HABP1). Unpaired two-tailed t-tests were performed (p<0.05) comparing the observed fibril widths between collagen functionalized with CBP-HABP1 and the others. Collagen+ (CBP-HABP1) fibrils were measured to be smaller than collagen-alone fibrils, resulting in a statistically significant difference. Though the average fibril width for CBP-HABP1 was observed to be less than that of the controls, it has a larger distribution of fibril sizes relative to the mean compared to collagen alone and collagen+ (CBP) (see Table 2). A distinct difference in the fibril width properties was observed when CBP and CBP-HABP1 were co-assembled with the collagen fibrils as compared to HABP1. This potentially implies similar molecular interactions are taking place between the those peptides (CBP and CBP-HABP1) and the collagen fibrils.

The fibril assembly with HABP1 exhibited wider variation of collagen fibril widths compared to collagen+ (CBP) or collagen+ (CBP-HABP1). The fibril width standard deviations observed on the collagen+ (CBP) and collagen+ (CBP-HABP1) samples were observed to be relatively similar compared to collagen+ (HABP1) (see Table 2). This finding evidences that there is a similar molecular interaction occurring between the collagen and CBP or CBP-HABP1, which differs in the case of collagen and HABP1.

TABLE 2
Statistics on fibril widths evaluated from AFM
topographical imaging.

| | | Collagen- | Collagen- | Collagen- |
|---|---|---|---|---|
| | Collagen | (CBP) | (HABP1) | (CBP-HABP1) |
| Mean (nm) | 98.89 | 121.40 | 129.90 | 82.37 |
| Standard | 19.26 | 20.66 | 87.643 | 26.47 |
| Deviation | | | | |
| Coefficient | 19.47 | 17.01 | 67.48 | 32.14 |
| of variation | | | | |

### Mechanical Properties of Peptide-Incorporated Collagen Platforms Pre/Post Mineralization

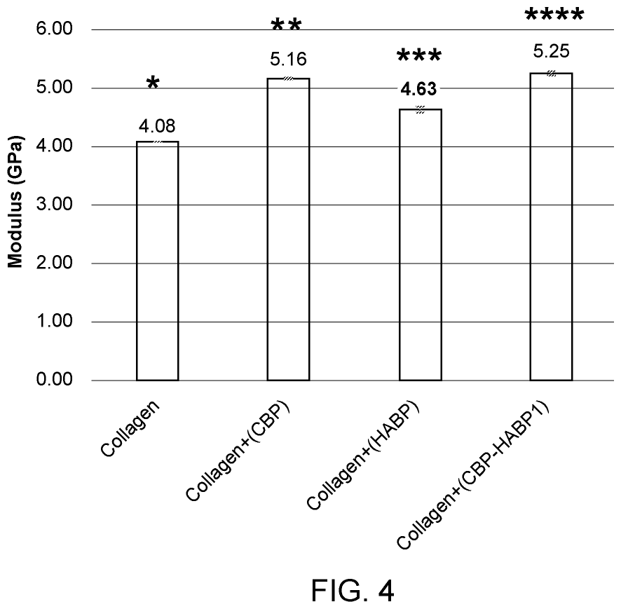

Building on the observed effects of the chimeric peptide on the collagen, next examined was if there was a change on the mechanical properties of the collagen due to the peptide presence. Quantitative modulus mapping was executed via PeakForce-QNM AFM to observe the changes in the elastic modulus distribution on the surfaces. The elastic modulus was previously established to be derived by using the Derjaguin-Muller-Toropov (DMT) model, and is thus reported as the DMT modulus [53]. Modulus mapping of the surface with peptides incorporated as compared to the collagen as the control were performed, where collagen alone shows a uniform, average elastic modulus, which is lower than those co-assembled with peptides as shown in FIG. 4. Comparatively, CBP or CBP-HABP1 incorporated into collagen resulted in values of 5.16 and 5.25 GPa, respectively. The relatively similar modulus values were observed to be much higher compared to collagen+ (HABP1) or collagen control samples (see FIG. 4). Collagen+ (CBP-HABP1) resulted in the largest increase in modulus compared to CBP or HABP1 peptides or collagen. The collagen+ (CBP-HABP1) also showed a larger surface modulus variance compared to that of collagen. These results suggest that peptide-dependent nanomechanical enhancement is plausible to explain the induced change in moduli for collagen fibril assembly.

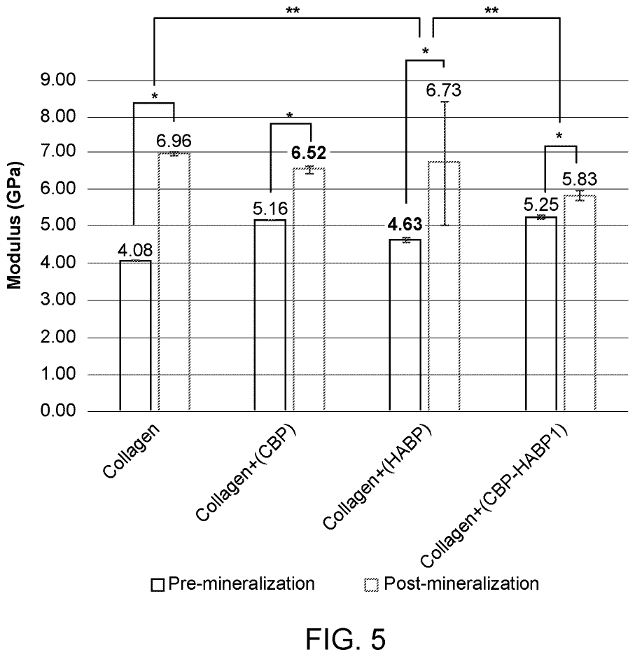

Next studied was the peptide-guided remineralization on the collagen platform using ALP-mediated mineralization. As a result of mineralization, major topographical and morphological changes were observed, resulting in rougher surfaces based upon AFM analysis. The topographical mineral characteristics mapped appeared to be significantly different between control and peptide-incorporated samples. These differences between mineral formations are subsequently reflected in the modulus mapping, where the average moduli examined show statistically significant changes between pre- and post-mineralization moduli per surface as shown in FIG. 5. Collagen alone showed the largest increase in elastic modulus, followed by collagen+ (HABP1) but which also exhibited the largest standard deviation. The larger moduli of collagen and collagen+ (HABP1) can be attributed to the potential supersaturation of mineral on the surface layer of collagen resulting in a non-uniform distribution of mineral deposition. This would lead to a greater variation in mineral formation at the substrate surface, as supported by the larger variance observed with HABP1 compared to the rest of peptide samples including collagen control. On the other hand, as a result of remineralization the elastic modulus of collagen+ (CBP) and collagen+ (CBP-HABP1) increased to 6.52 and 5.83 GPa, respectively, compared to the other samples. Although these values are lower than the modulus of HABP1 on collagen or control sample, the chimeric peptide (CBP-HABP1) had the smallest variations in mechanical properties, suggesting that it offered a more consistent route for directing peptide interactions within the collagen sample.

### Examining the Ca—P Deposits on the Peptide-Incorporated Collagen Platform

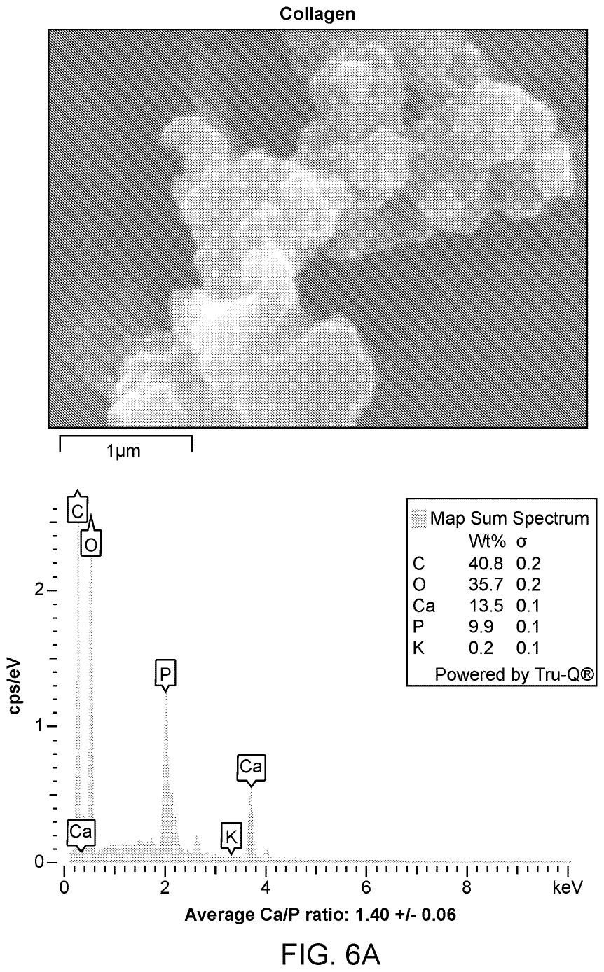

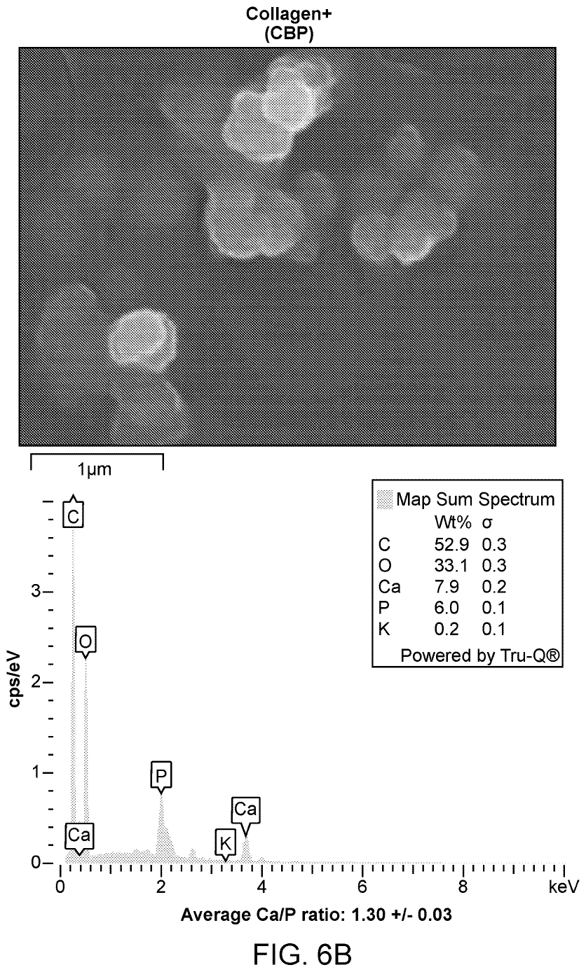

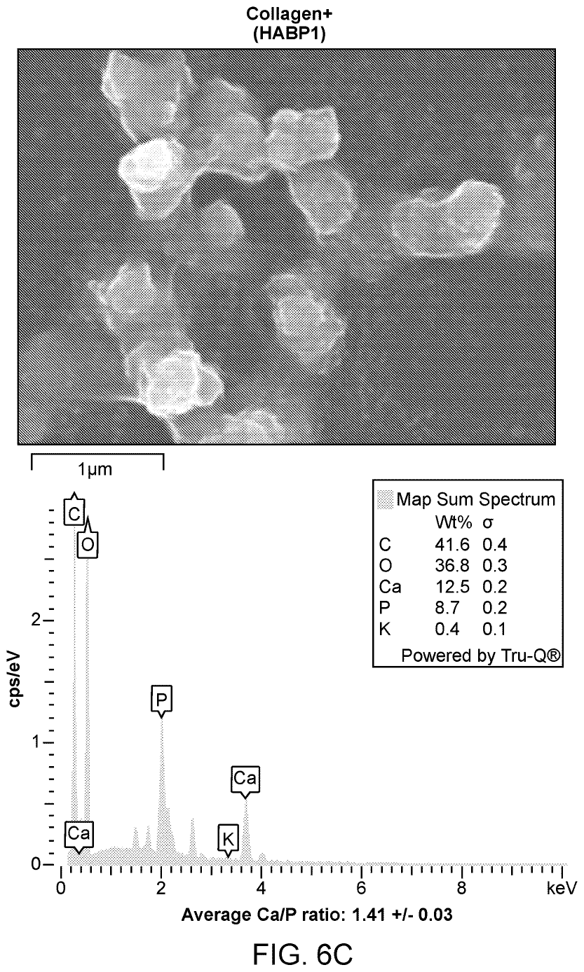

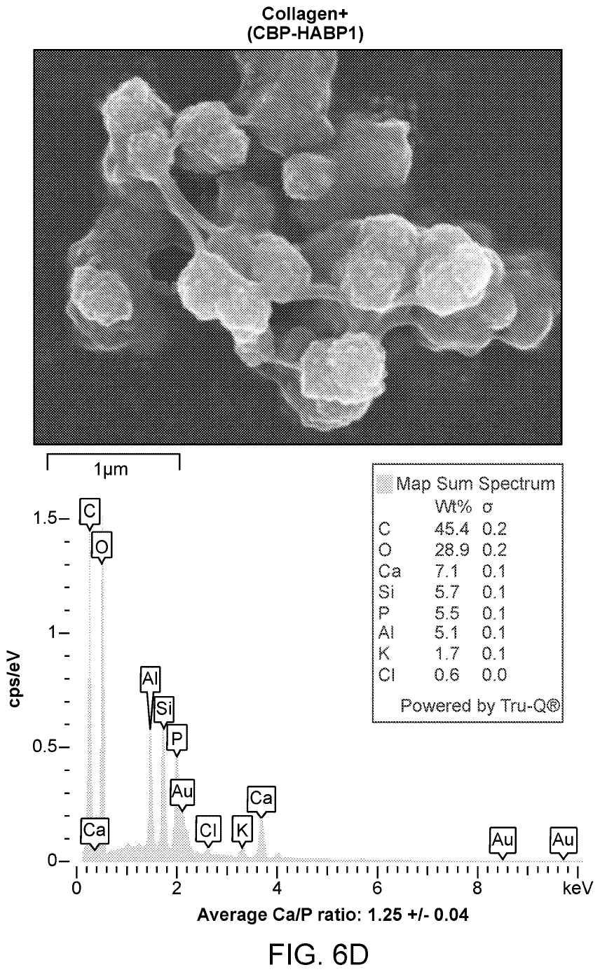

The composition and morphology of the Ca—P deposits formed on collagen samples were examined using scanning electron microscopy (SEM) with energy dispersive X-ray analysis (EDS). Incorporation of CBP-HABP1 to collagen resulted in similar morphology compared to the HABP1 incorporation alone, for which FIGS. 6A-6D summarize the results. The EDS results validated the finding of Ca—P mineral isomorphs on the peptide-incorporated collagen platforms. The calcium/phosphate ratios were calculated to examine compositional differences. The initial mineral formed in the collagen alone and collagen+ (HABP1) platforms had an average Ca—P ratio of 1.40 and 1.41, respectively. Whereas collagen+ (CBP) and collagen+ (CBP-HABP1) had comparatively lower average Ca—P ratios at 1.30 and 1.25, respectively. These values are similar to the molar ratio of octacalcium phosphate, which is around 1.33, and amorphous calcium phosphate (ACP), which is roughly 1.50 [54-57]. The incorporation of the collagen-binding motif resulted in a decrease in the Ca—P ratio to 1.25 of the mineral deposits directed by CBP-HABP1. This ratio suggest the presence of brushite (DCPD) and OCP.

Also performed was the spatial mapping of the mineral formed with or without peptide collagen samples. The surfaces were processed using the Multi-Otsu thresholding algorithm, separating surface and subsurface mineral formation along with any background noise. Comparing mineral formations on SEM images, collagen and collagen+ (CBP) both showed branched columnar mineral growth, whereas collagen+ (HABP1) and collagen+ (CBP-HABP1) showed more extensively branched columnar mineral growth as well as plate-like mineral growth. Based on the Multi-Otsu calculations, the CBP-HABP1-incorporated collagen samples showed similar mineral area coverage compared to HABP1-incorporated collagen samples. The histogram distribution of the CBP-HABP1 was also like that of HABP1 on collagen samples, indicating a similar mineral growth amount at their respective locations, compared to the collagen+ (CBP) or the control. This further supports the preservation of HABP1 mineralization activity in the chimeric peptide. These results suggest a robust mineral formation taking place with the inclusion of the chimeric CBP-HABP1 peptide when incorporated on collagen samples.

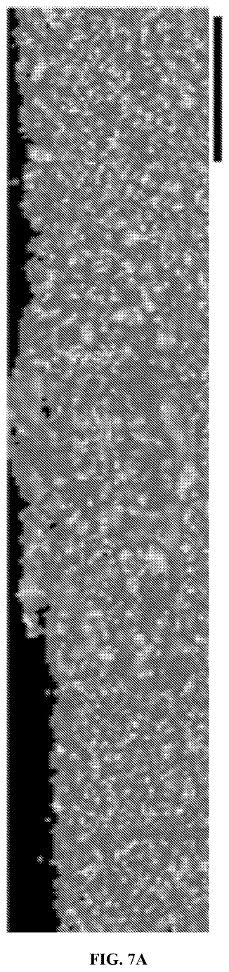

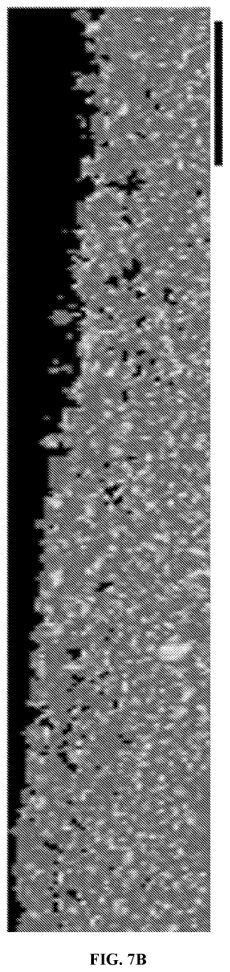

Next examined were the peptide-incorporated collagen samples following post-mineralization using the three-dimensional micro X-ray computed tomography (MicroXCT), shown in FIGS. 7A-7B. MicroXCT offers a non-destructive analysis of the internal structure with 3D imaging features and can be utilized as a physical indicator of intrafibrillar mineralization. The chimeric CBP-HABP1 peptide-incorporated collagen samples were examined post-mineralization and compared to the ALP-mineralized collagen samples. The shaded surface display (SSD) of the collagen control (FIG. 7B) and the collagen+ (CBP-HABP1) samples (FIG. 7A) were examined to isolate the mineral structure on the platform from the surrounding collagen within the acquired volume set of the sample. The mineralized collagen layer incorporated with CBP-HABP1 (FIG. 7A) showed denser formation compared to ALP-based mineralized collagen samples. The control collagen samples have clear voids areas distributed throughout the collagen interior regions (see FIG. 7B), whereas CBP-HABP1-incorporated collagen revealed almost no void areas (see FIG. 7A).

### Discussion

This results further support the use of the bifunctional peptides of the present technology as part of a self-strengthening dental adhesive to improve the integrity and durability of the adhesive/dentin (a/d) interface. While previous work of the inventors focused on improving the adhesive through the development of peptide-tethered-polymer systems [16] and self-strengthening adhesive formulations [1,15], the bifunctional peptides of the present technology (e.g., CBP-HABP1) are shown to enable collagen intrafibrillar mineralization to promote enclosure of demineralized collagen fibrils and help to improve the integrity and durability of the a/d interface.

CBP-HABP1 in particular exhibited conserved activity similar to its single domain collagen-binding motif on the collagen samples. AFM topography data showed similar fibril assembly with the incorporation of the CBP-HABP1 peptide compared to CBP—such topography was not observed with HABP1. The results with CBP-HABP1 and CBP can be attributed to the fact that both peptides contain the collagen-binding motif, thus likely bound to the collagen, in part, through sense-antisense domain interactions [58]. Whereas with HABP1, its non-specific interactions with the collagen fibrils may have attributed to the formation of larger fibril widths. Comparisons of dentin collagen fibrils formed in vivo were shown to have a diameter range of 80-100 nm that self-assemble in a hierarchical manner between second tier (microfibrillar) and third tier (fibrillar) levels [59,60]. Interestingly, the CBP-HABP1 peptide showed an average fibril diameter within the range observed for dentin [12].

The intermolecular interactions of collagen self-assembly mediated by CBP-HABP1 showed overall improvement in the nanomechanical properties of the collagen platform. While the CBP-HABP1 peptide aligns and molecularly interacts with the collagen, it also offers mineral deposition along these sites, via the hydroxyapatite-binding motif. The bifunctional peptide thus mimics tissue interfaces that promote intrafibrillar mineralization [52].

Early-stage mineral formation, prior to stages reaching octacalcium phosphate (molar Ca—P ratio, 1.33) has so far been minimally investigated. Here with the CBP-HABP1-integrated collagen platform, extensively branched columnar mineral growth as well as plate-like mineral morphologies were observed [60]. It is possible that a form of ACP is being observed when forming Ca—P minerals in aqueous environments, showing as low as 1.18 Ca—P molar ratios in early transient forms [57,61]. Mineral formations may also include DCPD as one of the possible apatite precursors in addition to ACP. In acidic environments, DCPD may undergo hydrolysis to more stable phases, and therefore it is widely utilized as bone cements.

The experiments disclosed and exemplary results presented herein thus evidence and support the bifunctional peptides and their use in the composition and methods of the present disclosure.

### REFERENCES

1. Ezazi, M.; Ye, Q.; Misra, A.; Tamerler, C.; Spencer, P. Autonomous-Strengthening Adhesive Provides Hydrolysis-Resistance and Enhanced Mechanical Properties in Wet Conditions. Molecules 2022, 27, 5505. https://doi.org/10.3390/molecules27175505.
2. Fischer, N. G.; Munchow, E. A.; Tamerler, C.; Bottino, M. C.; Aparicio, C. Harnessing biomolecules for bioinspired dental biomaterials. J. Mater. Chem. B 2020, 8, 8713-8747. https://doi.org/10.1039/d0tb01456g.
3. Spencer, P.; Ye, Q.; Kamathewatta, N. J. B.; Woolfolk, S. K.; Bohaty, B. S.; Misra, A.; Tamerler, C. Chemometrics-Assisted Raman Spectroscopy Characterization of Tunable Polymer-Peptide Hybrids for Dental Tissue Repair. Front. Mater. 2021, 8, 681415. https://doi.org/10.3389/fmats.2021.681415.
4. Ye, Q.; Spencer, P.; Yuca, E.; Tamerler, C. Engineered Peptide Repairs Defective Adhesive-Dentin Interface. Macromol. Mater. Eng. 2017, 302, 1600487. https://doi.org/10.1002/mame.201600487.
5. Melo, M. A.; Orrego, S.; Weir, M. D.; Xu, H. H.; Arola, D. D. Designing Multiagent Dental Materials for Enhanced Resistance to Biofilm Damage at the Bonded Interface. ACS Appl. Mater. Interfaces 2016, 8, 11779-11787. https://doi.org/10.1021/acsami.6b01923.
6. World Health Organization. Global Oral Health Status Report towards Universal Health Coverage for Oral Health by 2030; World Health Organization: Geneva, Switzerland, 2022.
7. Wen, P. Y. F.; Chen, M. X.; Zhong, Y. J.; Dong, Q. Q.; Wong, H. M. Global Burden and Inequality of Dental Caries, 1990 to 2019. J. Dent. Res. 2022, 101, 392-399. https://doi.org/10.1177/00220345211056247.
8. Song, L. Y.; Sarikaya, R.; Ye, Q.; Misra, A.; Tamerler, C.; Spencer, P. Multifunctional monomer acts as co-initiator and crosslinker to provide autonomous strengthening with enhanced hydrolytic stability in dental adhesives. Dent. Mater. 2020, 36, 284-295. https://doi.org/10.1016/j.dental.2019.11.007.
9. Hardan, L.; Daood, U.; Bourgi, R.; Cuevas-Suarez, C. E.; Devoto, W.; Zarow, M.; Jakubowicz, N.; Zamarripa-Calderon, J. E.; Radwanski, M.; Orsini, G.; et al. Effect of Collagen Crosslinkers on Dentin Bond Strength of Adhesive Systems: A Systematic Review and Meta-Analysis. Cells 2022, 11, 2417. https://doi.org/10.3390/cells11152417.
10. Spencer, P.; Ye, Q.; Song, L. Y.; Parthasarathy, R.; Boone, K.; Misra, A.; Tamerler, C. Threats to adhesive/dentin interfacial integrity and next generation bio-enabled multifunctional adhesives. J. Biomed. Mater. Res. B 2019, 107, 2673-2683. https://doi.org/10.1002/jbm.b.34358.
11. Iliev, G.; Hardan, L.; Kassis, C.; Bourgi, R.; Cuevas-Suarez, C. E.; Lukomska-Szymanska, M.; Mancino, D.; Haikel, Y.; Kharouf, N. Shelf Life and Storage Conditions of Universal Adhesives: A Literature Review. Polymers 2021, 13, 2708. https://doi.org/10.3390/polym13162708.
12. Goldberg, M.; Kulkarni, A. B.; Young, M.; Boskey, A. Dentin: Structure, composition and mineralization. Front. Biosci. 2011, 3, 711-735. https://doi.org/10.2741/e281.
13. Marshall, G. W., Jr. Dentin: Microstructure and characterization. Quintessence Int. 1993, 24, 606-617.
14. Sarikaya, R.; Ye, Q.; Song, L. Y.; Tamerler, C.; Spencer, P.; Misra, A. Probing the mineralized tissue-adhesive interface for tensile nature and bond strength. J. Mech. Behav. Biomed. 2021, 120, 104563. https://doi.org/10.1016/j.jmbbm.2021.104563.
15. Sarikaya, R.; Song, L. Y.; Yuca, E.; Xie, S. X.; Boone, K.; Misra, A.; Spencer, P.; Tamerler, C. Bioinspired multifunctional adhesive system for next generation bio-additively designed dental restorations. J. Mech. Behav. Biomed. Mater. 2021, 113, 104135. https://doi.org/10.1016/j.jmbbm.2020.104135.
16. Yuca, E.; Xie, S. X.; Song, L. Y.; Boone, K.; Kamathewatta, N.; Woolfolk, S. K.; Elrod, P.; Spencer, P.; Tamerler, C. Reconfigurable Dual Peptide Tethered Polymer System Offers a Synergistic Solution for Next Generation Dental Adhesives. Int. J. Mol. Sci. 2021, 22, 6552. https://doi.org/10.3390/ijms22126552.
17. Cheng, L.; Zhang, K.; Zhang, N.; Melo, M. A. S.; Weir, M. D.; Zhou, X. D.; Bai, Y. X.; Reynolds, M. A.; Xu, H. H. K. Developing a New Generation of Antimicrobial and Bioactive Dental Resins. J. Dent. Res. 2017, 96, 855-863. https://doi.org/10.1177/0022034517709739.
18. Mokeem, L. S.; Garcia, I. M.; Shahkarami, Y.; Blum, L.; Balhaddad, A. A.; Collares, F. M.; Williams, M. A.; Weir, M. D.; Melo, M. A. Core-shell nanostructures for improving dental restorative materials: A scoping review of composition, methods, and outcome. Smart. Mater. Med. 2023, 4, 102-110.
19. Munchow, E. A.; Bottino, M. C. Recent Advances in Adhesive Bonding—The Role of Biomolecules, Nanocompounds, and Bonding Strategies in Enhancing Resin Bonding to Dental Substrates. Curr. Oral. Health. Rep. 2017, 4, 215-227. https://doi.org/10.1007/s40496-017-0146-y.
20. Oestervemb, N. Bonding to Enamel and Dentin. In Textbook of Operative Dentistry, 2nd ed.; JP Medical Publishers: New Delhi, India, 2012.
21. Frassetto, A.; Breschi, L.; Turco, G.; Marchesi, G.; Di Lenarda, R.; Tay, F. R.; Pashley, D. H.; Cadenaro, M. Mechanisms of degradation of the hybrid layer in adhesive dentistry and therapeutic agents to improve bond durability—A literature review. Dent. Mater. 2016, 32, E41-E53. https://doi.org/10.1016/j.dental.2015.11.007.
22. Zhang, J. H.; Guo, X. W.; Zhang, X. M.; Wang, H. M.; Zhu, J. F.; Shi, Z. S.; Zhu, S.; Cui, Z. C. Hydrolysis-resistant and stress-buffering bifunctional polyurethane adhesive for durable dental composite restoration. Roy. Soc. Open. Sci. 2020, 7, 200457. https://doi.org/10.1098/rsos.200457.
23. Amin, F.; Fareed, M. A.; Zafar, M. S.; Khurshid, Z.; Palma, P. J.; Kumar, N. Degradation and Stabilization of Resin-Dentine Interfaces in Polymeric Dental Adhesives: An Updated Review. Coatings 2022, 12, 1094. https://doi.org/10.3390/coatings12081094.
24. De Brito, O. F. F.; de Oliveira, I. L. M.; Monteiro, G. Q. M. Hydrolytic and Biological Degradation of Bulk-fill and Self-adhering Resin Composites. Oper. Dent. 2019, 44, E223-E233. https://doi.org/10.2341/17-390-L.
25. Betancourt, D. E.; Baldion, P. A.; Castellanos, J. E. Resin-Dentin Bonding Interface: Mechanisms of Degradation and Strategies for Stabilization of the Hybrid Layer. Int. J. Biomater. 2019, 2019, 5268342. https://doi.org/10.1155/2019/5268342.
26. Reis, A.; Carrilho, M.; Breschi, L.; Loguercio, A. D. Overview of clinical alternatives to minimize the degradation of the resin-dentin bonds. Oper. Dent. 2013, 38, E1-E25. https://doi.org/10.2341/12-258-LIT.
27. Balhaddad, A. A. Toward dental caries: Exploring nanoparticle-based platforms and calcium phosphate compounds for dental restorative materials. Bioact. Mater. 2018, 4, 43-55.
28. Muliyar, S.; Shameem, K. A.; Thankachan, R. P.; Francis, P. G.; Jayapalan, C. S.; Hafiz, K. A. Microleakage in endodontics. J. Int. Oral Health 2014, 6, 99-104.
29. Zhang, K.; Zhang, N.; Weir, M. D.; Reynolds, M. A.; Bai, Y.; Xu, H. H. K. Bioactive Dental Composites and Bonding Agents Having Remineralizing and Antibacterial Characteristics. Dent. Clin. N. Am. 2017, 61, 669-687. https://doi.org/10.1016/j.cden.2017.05.002.
30. Moradian-Oldak, J.; George, A. Biomineralization of Enamel and Dentin Mediated by Matrix Proteins. J. Dent. Res. 2021, 100, 1020-1029. https://doi.org/10.1177/00220345211018405.
31. Wang, D.; Zhou, X.; Deng, J. R.; Fang, C. Q.; Wang, Z.; Wang, Q.; Huang, Y. W. Preparation and Characterization of Crystalline Hydroxyapatite Induced by Self-Assembled Peptide and the Potential Application in Remineralizing Dentin. Adv. Eng. Mater. 2021, 23, 2001470. https://doi.org/10.1002/adem.202001470.
32. Tian, Z. L.; Yu, S. Y.; Wang, H. M.; Yang, Y. B.; Zhu, X. Y.; Zhu, S. Biomimetic Mineralized Hydrophilic Polyurethane Primers for Inducing Dentin Tubule Fillings. Polymers 2022, 14, 4716. https://doi.org/10.3390/polym14214716.
33. Steinbauer, P.; Rohatschek, A.; Andriotis, O.; Bouropoulos, N.; Liska, R.; Thurner, P. J.; Baudis, S. Single-Molecule Force Spectroscopy Reveals Adhesion-by-Demand in Statherin at the Protein-Hydroxyapatite Interface. Langmuir 2020, 36, 13292-13300. https://doi.org/10.1021/acs.langmuir.0c02325.
34. Gungormus, M.; Fong, H.; Kim, I. W.; Evans, J. S.; Tamerler, C.; Sarikaya, M. Regulation of in vitro calcium phosphate mineralization by combinatorially selected hydroxyapatite-binding peptides. Biomacromolecules 2008, 9, 966-973. https://doi.org/10.1021/bm701037x.
35. Yuca, E.; Karatas, A. Y.; Seker, U. O. S.; Gungormus, M.; Dinler-Doganay, G.; Sarikaya, M.; Tamerler, C. In Vitro Labeling of Hydroxyapatite Minerals by an Engineered Protein. Biotechnol. Bioeng. 2011, 108, 1021-1030. https://doi.org/10.1002/bit.23041.
36. Niu, L. N.; Zhang, W.; Pashley, D. H.; Breschi, L.; Mao, J.; Chen, J. H.; Tay, F. R. Biomimetic remineralization of dentin. Dent. Mater. 2014, 30, 77-96. https://doi.org/10.1016/j.dental.2013.07.013.
37. Zhou, Z.; Ge, X. Y.; Bian, M. X.; Xu, T.; Li, N.; Lu, J. M.; Yu, J. H. Remineralization of dentin slices using casein phosphopeptide-amorphous calcium phosphate combined with sodium tripolyphosphate. Biomed. Eng. Online 2020, 19, 18. https://doi.org/10.1186/s12938-020-0756-9.
38. Sattabanasuk, V.; Burrow, M. F.; Shimada, Y.; Tagami, J. Bonding of resin luting cements to dentine after casein phosphopeptide-amorphous calcium phosphate (CPP-ACP) treatment. Int. J. Adhes. Adhes. 2014, 54, 93-99. https://doi.org/10.1016/j.ijadhadh.2014.05.008.
39. de Sousa, J. P.; Carvalho, R. G.; Barbosa-Martins, L. F.; Torquato, R. J. S.; Mugnol, K. C. U.; Nascimento, F. D.; Tersariol, I. L. S.; Puppin-Rontani, R. M. The Self-Assembling Peptide P11-4 Prevents Collagen Proteolysis in Dentin. J. Dent. Res. 2019, 98, 347-354. https://doi.org/10.1177/0022034518817351.
40. Li, Z.; Ren, Q.; Han, S.; Ding, L.; Qin, X.; Hu, D.; He, T.; Tian, T.; Lu, Z.; Zhang, L. Promoting effect of a calcium-responsive self-assembly beta-sheet peptide on collagen intrafibrillar mineralization. Regen. Biomater. 2022, 9, rbac059. https://doi.org/10.1093/rb/rbac059.
41. Xia, K.; Chen, Z.; Chen, J.; Xu, H. X.; Xu, Y. F.; Yang, T.; Zhang, Q. RGD- and VEGF-Mimetic Peptide Epitope-Functionalized Self-Assembling Peptide Hydrogels Promote Dentin-Pulp Complex Regeneration. Int. J. Nanomed. 2020, 15, 6631-6647. https://doi.org/10.2147/Ijn.S253576.
42. Mukherjee, K.; Visakan, G.; Phark, J. H.; Moradian-Oldak, J. Enhancing Collagen Mineralization with Amelogenin Peptide: Toward the Restoration of Dentin. ACS Biomater. Sci. Eng. 2020, 6, 2251-2262. https://doi.org/10.1021/acsbiomaterials.9b01774.
43. Gungormus, M.; Ozdogan, M. S.; Ertem, S. Y.; Tulumbaci, F.; Kara, H.; Orhan, M. Accelerated Calcium Phosphate Mineralization by Peptides with Adjacent Oppositely Charged Residues. ACS Biomater. Sci. Eng. 2020, 6, 3791-3798. https://doi.org/10.1021/acsbiomaterials.0c00194.
44. Gungormus, M.; Tulumbaci, F. Peptide-assisted pre-bonding remineralization of dentin to improve bonding. J. Mech. Behav. Biomed. 2021, 113, 104119. https://doi.org/10.1016/j.jmbbm.2020.104119.
45. Bermudez, M.; Hoz, L.; Montoya, G.; Nidome, M.; Perez-Soria, A.; Romo, E.; Soto-Barreras, U.; Garnica-Palazuelos, J.; Aguilar-Medina, M.; Ramos-Payan, R.; et al. Bioactive Synthetic Peptides for Oral Tissues Regeneration. Front. Mater. 2021, 8, 655495. https://doi.org/10.3389/fmats.2021.655495.
46. Lin, S.-Y.; Li, M.-J.; Cheng, W.-T. FT-IR and Raman vibrational microspectroscopies used for spectral biodiagnosis of human tissues. Spectroscopy 2007, 21, 278765. https://doi.org/10.1155/2007/278765.
47. Oladepo, S. A.; Xiong, K.; Hong, Z. M.; Asher, S. A.; Handen, J.; Lednev, I. K. UV Resonance Raman Investigations of Peptide and Protein Structure and Dynamics. Chem. Rev. 2012, 112, 2604-2628. https://doi.org/10.1021/cr200198a.
48. KONG, J.; YU, S. Fourier Transform Infrared Spectroscopic Analysis of Protein Secondary Structures. Acta Biochim. Biophys. Sin. 2007, 39, 549-559. https://doi.org/10.1111/j.1745-7270.2007.00320.x.
49. Lamiable, A.; Thevenet, P.; Rey, J.; Vavrusa, M.; Derreumaux, P.; Tuffery, P. PEP-FOLD3: Faster denovo structure prediction for linear peptides in solution and in complex. Nucleic Acids Res. 2016, 44, W449-W454. https://doi.org/10.1093/nar/gkw329.
50. Meng, E. C.; Pettersen, E. F.; Couch, G. S.; Huang, C. C.; Ferrin, T. E. Tools for integrated sequence-structure analysis with UCSF Chimera. BMC Bioinform. 2006, 7, 339.
51. Sreerama, N.; Woody, R. W. Estimation of protein secondary structure from circular dichroism spectra: Comparison of CONTIN, SELCON, and CDSSTR methods with an expanded reference set. Anal. Biochem. 2000, 287, 252-260. https://doi.org/10.1006/abio.2000.4880.
52. Hafner, A. E.; Gyori, N. G.; Bench, C. A.; Davis, L. K.; Saric, A. Modeling Fibrillogenesis of Collagen-Mimetic Molecules. Biophys. J. 2020, 119, 1791-1799. https://doi.org/10.1016/j.bpj.2020.09.013.
53. Derjaguin, B. V.; Muller, V. M.; Toporov, Y. P. Effect of Contact Deformations on the Adhesion of Particles. Prog. Surf. Sci. 1994, 45, 131-143. https://doi.org/10.1016/0079-6816(94)90044-2.
54. Woolfolk, S. K.; Cloyd, A. K.; Ye, Q.; Boone, K.; Spencer, P.; Snead, M. L.; Tamerler, C. Peptide-Enabled Nanocomposites Offer Biomimetic Reconstruction of Silver Diamine Fluoride-Treated Dental Tissues. Polymers 2022, 14, 1368. https://doi.org/10.3390/polym14071368.
55. LeGeros, R. Z. Calcium Phosphate-Based Osteoinductive Materials. Chem. Rev. 2008, 108, 4742-4753. https://doi.org/10.1021/cr800427g.
56. Sheikh, Z.; Abdallah, M. N.; Hanafi, A. A.; Misbahuddin, S.; Rashid, H.; Glogauer, M. Mechanisms of in Vivo Degradation and Resorption of Calcium Phosphate Based Biomaterials. Materials 2015, 8, 7913-7925. https://doi.org/10.3390/ma8115430.
57. Meyer, F.; Amaechi, B. T.; Fabritius, H. O.; Enax, J. Overview of Calcium Phosphates used in Biomimetic Oral Care. Open Dent. J. 2018, 12, 406-423. https://doi.org/10.2174/1874210601812010406.
58. Desouza, S. J.; Brentani, R. Collagen Binding-Site in Collagenase Can Be Determined Using the Concept of Sense-Antisense Peptide Interactions. J. Biol. Chem. 1992, 267, 13763-13767.
59. Bertassoni, L. E.; Swain, M. V. Removal of dentin non-collagenous structures results in the unraveling of microfibril bundles in collagen type I. Connect Tissue Res. 2017, 58, 414-423. https://doi.org/10.1080/03008207.2016.1235566.
60. Habelitz, S.; Balooch, M.; Marshall, S. J.; Balooch, G.; Marshall, G. W. In situ atomic force microscopy of partially demineralized human dentin collagen fibrils. J. Struct. Biol. 2002, 138, 227-236. https://doi.org/10.1016/S1047-8477(02)00029-1.
61. Dorozhkin, S. V.; Epple, M. Biological and medical significance of calcium phosphates. Angew. Chem. Int. Edit. 2002, 41, 3130-3146. https://doi.org/10.1002/1521-3773(20020902)41: 17<3130::Aid-Anie3130>3.0.Co;2-1.
62. Scharnow, A. M.; Solinski, A. E.; Wuest, W. M. Targeting S. mutans biofilms: A perspective on preventing dental caries. MedChemComm 2019, 10, 1057-1067. https://doi.org/10.1039/c9md00015a.
63. Jayaraj, D.; Ganesan, S. Salivary pH and Buffering Capacity as Risk Markers for Early Childhood Caries: A Clinical Study. Int. J. Clin. Pediatr. Dent. 2015, 8, 167-171. https://doi.org/10.5005/jp-journals-10005-1307.
64. Vahedi, M.; Davoodi, P.; Goodarzi, M.; Rezaei-Soufi, L.; Jazaeri, M.; Mortazavi, H.; Moghimbeigi, E. Comparison of Salivary Ion Activity Product for Hydroxyapatite (IPHA), Alkaline Phosphatase and Buffering Capacity of Adults According to Age and Caries Severity. J. Dent. 2012, 13, 139-145.
65. Hegde, M. N.; Tahiliani, D.; Shetty, S.; Devadiga, D. Salivary alkaline phosphatase and calcium in caries-active type II diabetes mellitus patients: An in vivo study. Contemp. Clin. Dent. 2014, 5, 440-444. https://doi.org/10.4103/0976-237X.142805.
66. Iijima, M.; Moriwaki, Y.; Yamaguchi, R.; Kuboki, Y. Effect of solution pH on the calcium phosphates formation and ionic diffusion on and through the collagenous matrix. Connect. Tissue Res. 1997, 36, 73-83. https://doi.org/10.3109/03008209709160215.
67. Buzalaf, M. A. R.; Hannas, A. R.; Magalhaes, A. C.; Rios, D.; Honorio, H. M.; Delbem, A. C. B. pH-cycling models for in vitro evaluation of the efficacy of fluoridated dentifrices for caries control: Strengths and limitations. J. Appl. Oral. Sci. 2010, 18, 316-334. https://doi.org/10.1590/S1678-77572010000400002.
68. Tsujimoto, A.; Barkmeier, W. W.; Teixeira, E. C.; Takamizawa, T.; Miyazaki, M.; Latta, M. A. Fatigue bond strength of dental adhesive systems: Historical background of test methodology, clinical considerations and future perspectives. Jpn. Dent. Sci. Rev. 2022, 58, 193-207. https://doi.org/10.1016/j.jdsr.2022.05.001.
69. Blalock, J. E.; Smith, E. M. Hydropathic Anti-Complementarity of Amino-Acids Based on the Genetic-Code. Biochem. Bioph. Res. Commun. 1984, 121, 203-207. https://doi.org/10.1016/0006-291x(84)90707-1.
70. Brentani, R. R. Biological Implications of Complementary Hydropathy of Amino-Acids. J. Theor. Biol. 1988, 135, 495-499. https://doi.org/10.1016/S0022-5193(88)80272-8.
71. Brentani, R. R. Complementary Hydropathy and the Evolution of Interacting Polypeptides. J. Mol. Evol. 1990, 31, 239-243. https://doi.org/10.1007/Bf02109501.

While certain embodiments have been illustrated and described, it should be understood that changes and modifications may be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations may be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which may of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A bifunctional peptide of amino acid sequence

TKKLTLRT-X₁-MLPHHGA (SEQ ID NO: 1)
or a pharmaceutically acceptable salt thereof and/or a solvate thereof,
wherein X₁ is absent or a spacer of 1, 2, 3, 4, 5, 6, 7, 8, 9, or 10 amino acid residues.
B. The bifunctional peptide of Paragraph A, wherein X₁ is selected from EAAAK (SEQ ID NO: 2), APA, GGG, PAPAP (SEQ ID NO: 5), GSGGG (SEQ ID NO: 6), KGSVLSA (SEQ ID NO: 7), PKSALQEL (SEQ ID NO: 8), GLALLGWG (SEQ ID NO: 9), LGWLSAV (SEQ ID NO: 10), WLMNYFWPL (SEQ ID NO: 11), and YLMNYLLPY (SEQ ID NO: 12).
C. The bifunctional peptide of Paragraph A or Paragraph B, wherein the bifunctional peptide is TKKLTLRTEAAAKMLPHHGA (SEQ ID NO: 13).
D. The bifunctional peptide of Paragraph A or Paragraph B, wherein the bifunctional peptide is TKKLTLRTAPAMLPHHGA (SEQ ID NO: 14).
E. The bifunctional peptide of Paragraph A or Paragraph B, wherein the bifunctional peptide is TKKLTLRTGGGMLPHHGA (SEQ ID NO: 15).
F. The bifunctional peptide of Paragraph A or Paragraph B, wherein the bifunctional peptide is TKKLTLRTPAPAPMLPHHGA (SEQ ID NO: 16).
G. The bifunctional peptide of Paragraph A or Paragraph B, wherein the bifunctional peptide is TKKLTLRTGSGGGMLPHHGA (SEQ ID NO: 17).
H. The bifunctional peptide of Paragraph A or Paragraph B, wherein the bifunctional peptide is TKKLTLRTKGSVLSAMLPHHGA (SEQ ID NO: 18).
I. The bifunctional peptide of Paragraph A or Paragraph B, wherein the bifunctional peptide is TKKLTLRTPKSALQELMLPHHGA (SEQ ID NO: 19).
J. The bifunctional peptide of Paragraph A or Paragraph B, wherein the bifunctional peptide is TKKLTLRTGLALLGWGMLPHHGA (SEQ ID NO: 20).
K. The bifunctional peptide of Paragraph A or Paragraph B, wherein the bifunctional peptide is TKKLTLRTLGWLSAVMLPHHGA (SEQ ID NO: 21).
L. The bifunctional peptide of Paragraph A or Paragraph B, wherein the bifunctional peptide is TKKLTLRTWLMNYFWPLMLPHHGA (SEQ ID NO: 22).
M. The bifunctional peptide of Paragraph A or Paragraph B, wherein the bifunctional peptide is TKKLTLRTYLMNYLLPYMLPHHGA (SEQ ID NO: 23).
N. The bifunctional peptide of Paragraph A, wherein the bifunctional peptide is TKKLTLRTMLPHHGA (SEQ ID NO: 24).
O. A composition comprising
   a bifunctional peptide of any one of Paragraphs A-N; and
   a pharmaceutically acceptable carrier.
P. The composition of Paragraph O, wherein the composition comprises an effective amount of the bifunctional peptide for treating a dental surface.
Q. The composition of Paragraph O or Paragraph P, wherein the bifunctional peptide is present at a concentration of about 20 μM to about 150 μM.
R. A dental adhesive composition comprising
   a bifunctional peptide of any one of Paragraphs A-N; and
   one or more dental adhesives.
S. The composition of Paragraph R, wherein the dental adhesive composition comprises an effective amount of the bifunctional peptide for treating a dental surface.
T. The composition of Paragraph R or Paragraph S, wherein the bifunctional peptide is present at a concentration of about 20 μM to about 150 μM.
U. A method of adhering a dental composite to a dental surface, the method comprising:
   administering to the dental surface a bifunctional peptide of any one of Paragraphs A-N, a composition of any one of Paragraphs O-Q, and/or a dental adhesive composition of any one of Paragraphs R-T to provide a treated dental surface; and
   contacting the dental composite with the treated dental surface.
V. The method of Paragraph U, wherein the administering comprises administering an effective amount of the bifunctional peptide for treating the dental surface
W. The method of Paragraph U or Paragraph V, wherein the dental surface is a dental enamel and/or dentin.
X. The method of Paragraph W, wherein the dental enamel and/or dentin comprises a carious region, a hypomineralized region, or both a carious region and a hypomineralized region.
Y. The method of any one of Paragraphs U-X, wherein administering comprises contacting the dental surface with the bifunctional peptide, the composition, and/or the dental adhesive composition for about 10 seconds to about 4 hours.
Z. The method of any one of Paragraphs U-Y, wherein the method adds new mineral to the dental surface.
AA. The method of any one of Paragraphs U-Z, wherein the method provides collagen infiltration from the dental surface to the dental composite.
Other embodiments are set forth in the following claims, along with the full scope of equivalents to which such claims are entitled.

1. A bifunctional peptide of amino acid sequence
TKKLTLRT-X₁-MLPHHGA (SEQ ID NO: 1)
or a pharmaceutically acceptable salt thereof and/or a solvate thereof,
wherein X₁ is absent or a spacer of 1, 2, 3, 4, 5, 6, 7, 8, 9, or 10 amino acid residues.

2. The bifunctional peptide of claim 1, wherein X₁ is selected from EAAAK (SEQ ID NO: 2), APA, GGG, PAPAP (SEQ ID NO: 5), GSGGG (SEQ ID NO: 6), KGSVLSA (SEQ ID NO: 7), PKSALQEL (SEQ ID NO: 8), GLALLGWG (SEQ ID NO: 9), LGWLSAV (SEQ ID NO: 10), WLMNYFWPL (SEQ ID NO: 11), and YLMNYLLPY (SEQ ID NO: 12).

3. The bifunctional peptide of claim 1, wherein the bifunctional peptide is TKKLTLRTEAAAKMLPHHGA (SEQ ID NO: 13).

4. The bifunctional peptide of claim 1, wherein the bifunctional peptide is TKKLTLRTAPAMLPHHGA (SEQ ID NO: 14).

5. The bifunctional peptide of claim 1, wherein the bifunctional peptide is TKKLTLRTGGGMLPHHGA (SEQ ID NO: 15).

6. The bifunctional peptide of claim 1, wherein the bifunctional peptide is TKKLTLRTPAPAPMLPHHGA (SEQ ID NO: 16).

7. The bifunctional peptide of claim 1, wherein the bifunctional peptide is TKKLTLRTGSGGGMLPHHGA (SEQ ID NO: 17).

8. The bifunctional peptide of claim 1, wherein the bifunctional peptide is TKKLTLRTKGSVLSAMLPHHGA (SEQ ID NO: 18).

9. The bifunctional peptide of claim 1, wherein the bifunctional peptide is TKKLTLRTPKSALQELMLPHHGA (SEQ ID NO: 19).

10. The bifunctional peptide of claim 1, wherein the bifunctional peptide is TKKLTLRTGLALLGWGMLPHHGA (SEQ ID NO: 20).

11. The bifunctional peptide of claim 1, wherein the bifunctional peptide is TKKLTLRTLGWLSAVMLPHHGA (SEQ ID NO: 21).

12. The bifunctional peptide of claim 1, wherein the bifunctional peptide is TKKLTLRTWLMNYFWPLMLPHHGA (SEQ ID NO: 22).

13. The bifunctional peptide of claim 1, wherein the bifunctional peptide is TKKLTLRTYLMNYLLPYMLPHHGA (SEQ ID NO: 23).

14. The bifunctional peptide of claim 1, wherein the bifunctional peptide is TKKLTLRTMLPHHGA (SEQ ID NO: 24).

15. A composition comprising
a bifunctional peptide of claim 1; and
a pharmaceutically acceptable carrier.

16. The composition of claim 15, wherein the composition comprises an effective amount of the bifunctional peptide for treating a dental surface.

17. The composition of claim 15, wherein the bifunctional peptide is present at a concentration of about 20 μM to about 150 μM.

18. A dental adhesive composition comprising
a bifunctional peptide of claim 1; and
one or more dental adhesives.

19. (canceled)

20. The composition of claim 18, wherein the bifunctional peptide is present at a concentration of about 20 μM to about 150 μM.

21. A method of adhering a dental composite to a dental surface, the method comprising:
administering to the dental surface a bifunctional peptide of claim 1 to provide a treated dental surface; and
contacting the dental composite with the treated dental surface.

22.-27. (canceled)
